(19) United States
(12) Patent Application Publication
(10) Pub. No.: US 2026/0204377 A1
(43) Pub. Date: Jul. 16, 2026

(54) METHOD AND SYSTEM FOR DETERMINING AIRWAY AND LUNG DEPOSITION OF AN INHALED SUBSTANCE

(71) Applicant: FLUIDDA RESPI NV, Kontich (BE)

(72) Inventors: Jan DE BACKER, Atlanta, GA (US); Hosein SADAFI, Aintree (AU)

(73) Assignee: FLUIDDA RESPI NV, Kontich (BE)

(21) Appl. No.: 19/132,721

(22) PCT Filed: Dec. 6, 2023
(86) PCT No.: PCT/EP2023/084478

§ 371 Date: May 23, 2025

(30) Foreign Application Priority Data
Dec. 6, 2022 (EP) 22211833.3

(51) Int. Cl. G16H 20/10; G16H 10/60; G16H 50/20

(52) U.S. Cl. CPC G16H 20/10; G16H 10/60; G16H 50/20

(57) ABSTRACT

Provided is a computer implemented method for determining airway deposition of an inhaled substance in a zone of a lung of a subject, comprising: —receiving a patient dataset of the subject comprising a parameter set of two or more parameters and corresponding values, wherein the parameters in the parameter set form at least four dimensionless numbers in a dimensionless correlation specific to the zone, —determining, from the dimensionless correlation and the corresponding values of the parameters in the parameter set, the airway deposition of the inhaled substance in the zone of the lung of the subject.

### FIELD OF THE INVENTION

The present invention is in a field of determining airway deposition of an inhaled substance.

### BACKGROUND TO THE INVENTION

Inhaled substances are used in clinical practice for treatment of diseases such as asthma or chronic obstructive pulmonary disease. Effectiveness of treatment is related to the airway deposition, namely, the zone(s) of deposition, and the quantity of deposition in each zone.

In vivo scintigraphy is the gold standard for assessing airway deposition (Carvalho et al, Int J Pharm, 2011; 406: 1-10; Conway J. Adv Drug Deliv Rev 2012; 64: 357-368), however, it exposes the subject to radioactively labelled compounds, and it needs to be repeated for each different formulation.

Computational fluid dynamics (CFD) may be used to predict deposition of inhaled substances. However, they can be computationally expensive because they simulate airflow within 3D models of the lung (e.g. Van Holsbeke et al, Ther Adv Respir Dis 2018, Vol. 12: 1-15).

A new method for determining airway deposition is needed that is computationally less expensive, as accurate as existing methods, patient specific, and takes into account different formulations and inhalation flows.

### SUMMARY OF THE INVENTION

Provided herein is a computer implemented method for determining airway deposition of an inhaled substance in a zone of a lung of a subject, comprising:
receiving a patient dataset of the subject comprising a parameter set of two or more parameters and corresponding values, wherein the parameters in the parameter set form at least four dimensionless numbers in a dimensionless correlation specific to the zone,
determining, from the dimensionless correlation and the corresponding values of the parameters in the parameter set, the airway deposition of the inhaled substance in the zone of the lung of the subject.

According to a preferred aspect:
a first component of the dimensionless correlation is a dimensionless number (π_{0,zone}) representative of the airway deposition for the zone being determined, and
a second component of the dimensionless correlation is a dimensionless group comprising the at least four dimensionless numbers for the zone being determined.

According to a preferred aspect, the dimensionless correlation has a form of Eq. 1:

$${π}_{0 , zone} = {c}_{0} ( {π}_{1}^{c 1} \times {π}_{2}^{c 2} \times {π}_{3}^{c 3} \times … \times {π}_{m}^{c m} ) , \qquad [ Eq . 1 ]$$

where
π_{0,zone} is a dimensionless number that is deposition in the zone (first component),
c₀ (π₁^{c1}×π₂^{c2}×π₃^{c3}× . . . ×πₘ^{cm}) is the dimensionless group (second component),
each π _{of} π_{1 to m} is a dimensionless number for the zone,
each c of c1 to cm is an exponent of the dimensionless number,
m is at least 4, between 4 and 20, preferably between 4 and 9, more preferably 4, 5 or 6,
c₀ is a multiplier of the correlation,
each of π of π_{1 to} πₘ, each c of c1 to cm, and c₀ for the zone is determined from multiple patient record datasets and regression analysis.

According to a preferred aspect, each of π of π₁ to πₘ, each c of c1 to cm, and c₀ for the zone is determined by:
populating the dimensionless group of Eq. 1 with 6 or more, preferably 6 to 20 dimensionless numbers selected from a pool of dimensionless numbers,
performing multiple rounds regression analysis, wherein each round comprises:
   adjusting values for c₀, c1 to cm until there is convergence to a solution for deposition for the multiple patient record datasets,
   substituting a portion (e.g. 2 to 5) of the dimensionless numbers in the dimensionless group with different dimensionless numbers selected from the pool of dimensionless numbers, and
repeating the adjusting and substituting until improvement in convergence (interclass correlation) is minimal.

According to a preferred aspect, the pool of dimensionless numbers comprises:

$$\frac{{A}_{b r a n c h}}{{A}_{trachea}} , FPF , \frac{{L}_{c h}}{MMAD} , \hat{L} , LNParam , \frac{{Q}_{max}}{{Q}_{i n h}} , \frac{Rate \cdot {( {t}_{i n j} )}^{2}}{T {y}_{i n h}} , Re , stdLN , \hat{t} ,$$

$${V}_{b r a n c h , n o r m a l} , Vent , β , St , \frac{{TV}_{i n h}}{{V}_{b r a n c h}} , R e , F r , \frac{{A}_{b r a n c h}}{{( {L}_{c h} )}^{2}} , {N}_{outlet} , {V}_{TLC , normal} ,$$

$$\frac{{V}_{FRC}}{{V}_{TLC}} , sin ( α ) , \hat{u} , \frac{{V}_{b r a n c h}}{{V}_{TLC}} , G r , \frac{{A}_{inlet}}{{( {L}_{c h} )}^{2}} , \frac{{L}_{c h}}{MMAD} , We , Eo , \frac{{d}_{n o z z l e}^{2}}{{A}_{inlet}} , IE , RR ,$$

$$Br , s , and Tor .$$

According to a preferred aspect, the dimensionless numbers of Eq. 1 are selected from:

$$\frac{{A}_{b r a n c h}}{{A}_{trachea}} , FPF , \frac{{L}_{c h}}{MMAD} , \hat{L} , LNParam , \frac{{Q}_{max}}{{Q}_{i n h}} , \frac{Rate \cdot {( {t}_{i n j} )}^{2}}{{TV}_{i n h}} , Re , stdLN , \hat{t} ,$$

$${V}_{b r a n c h , n o r m a l} , Vent , and β .$$

According to a preferred aspect, the parameters in the parameter set and the dimensionless numbers comprise those of Table AA1, selected according to the zone of the lung of the subject where airway deposition is to be determined:

TABLE AA1

| Zone | Parameters | DN |
|---|---|---|
| Intrathoracic, IT | FPF, GSD, L_{ch}, MMAD, Qₘₐₓ, Qᵢₙₕ | $FPF , \frac{{Q}_{ma x}}{{Q}_{i n h}} , LNParam , \frac{{L}_{c h}}{MMAD}$ |
| Peripheral, PR | A_{trachea}, FPF, L_{ch}, MMAD, Qₘₐₓ, Qᵢₙₕ, Rate, u, ρₐ, μₐ | $FPF , \frac{{Q}_{m ax}}{{Q}_{i n h}} , R e , β$ |
| Distal, DI | FPF, GSD, L_{ch}, L_{ch,ref}, MMAD, Qₘₐₓ, Qᵢₙₕ | $FPF , stdLN , \hat{L} , \frac{{Q}_{ma x}}{{Q}_{i n h}}$ |
| Right lower | FPF, L_{ch}, L_{ch,ref}, | FPF, V_{branch,normal}, \hat{L}, \hat{t} |
| lobe, RLL | tᵢₙⱼ, tᵢₙₕ, V_{branch}, V_{branch,total} | |
| Left lower lobe, LLL | FPF tᵢₙⱼ, Rate, TVᵢₙₕ, V_{branch,zone}, V_{branch,total}, V_{TLC}, V_{FRC}, | $FPF , Vent , {V}_{branch , normal} , \frac{Rate \cdot {( {t}_{inj} )}^{2}}{T {V}_{i n h}}$ |
| | V_{FRC,total}, V_{TLC,total} | |
| Right upper lobe, RUL | FPF, Qₘₐₓ, Qᵢₙₕ, V_{TLC}, V_{FRC}, V_{FRC,total}, V_{TLC,total}, V_{branch}, V_{branch,total} | $FPF , Vent , \frac{{Q}_{ma x}}{{Q}_{i n h}} , {V}_{branch , normal}$ |
| Left upper lobe, LUL | A_{branch}, A_{trachea}, FPF, L_{ch}, MMAD, Qₘₐₓ, Qᵢₙₕ | $FPF , \frac{{Q}_{ma x}}{{Q}_{i n h}} , \frac{{L}_{c h}}{MMAD} , \frac{{A}_{branch}}{{A}_{trachea}}$ |
| Right middle lobe, RML | A_{branch}, A_{trachea}, FPF, MMAD, u, V_{TLC}, V_{FRC}, V_{TLC,total}, ρₐ, μₐ | $FPF , Vent , R e , \frac{{A}_{branch}}{{A}_{trachea}}$ |
| Extrathoracic, ET | FPF, GSD, L_{ch}, MMAD, Qₘₐₓ, Qᵢₙₕ | $FPF , \frac{{Q}_{m ax}}{{Q}_{i n h}} , LNParam , \frac{{L}_{c h}}{MMAD}$ |
| Total lobes, TL | FPF, L_{ch}, MMAD, Qₘₐₓ, Qᵢₙₕ, V_{branch}, V_{branch,total} | $FPF , \frac{{Q}_{ma x}}{{Q}_{i n h}} , {V}_{branch , normal} , \frac{{L}_{c h}}{MMAD}$ |

According to a preferred aspect:
the zone is the IT zone,

$${π}_{0 , zone} = {c}_{0} ( {( FPF )}^{c 1} \times {( \frac{{Q}_{max}}{{Q}_{i n h}} )}^{c 2} \times {( LNParam )}^{c 3} \times {( \frac{{L}_{c h}}{MMAD} )}^{c 4} … \times {π}_{m}^{c m} ) , \qquad Eq . 1 is$$

c1>c2>c3>c4>cm, wherein terms where m>4 are optionally present.

According to a preferred aspect:
the zone is the PR zone,

$${π}_{0 , zone} = {c}_{0} ( {( FPF )}^{c 1} \times {( \frac{{Q}_{max}}{{Q}_{i n h}} )}^{c 2} \times {( Re )}^{c 3} \times {( β )}^{c 4} … \times {π}_{m}^{c m} ) , \qquad Eq . 1 is$$

c1>c2>c3>c4>cm, wherein terms where m>4 are optionally present.

According to a preferred aspect:
the zone is the DI zone,

$${π}_{0 , zone} = {c}_{0} ( {( FPF )}^{c 1} \times {( stdLN )}^{c 2} \times {( \hat{L} )}^{c 3} \times {( \frac{{Q}_{max}}{{Q}_{i n h}} )}^{c 4} … \times {π}_{m}^{c m} ) , \qquad Eq . 1 is$$

and
c1>c2>c3>c4>cm, wherein terms where m>4 are optionally present.

According to a preferred aspect:
the zone is the RLL zone,

$$Eq . 1 is : {π}_{0 , zone} = {c}_{0} ( {( F P F , )}^{c 1} \times {( {V}_{branch , normal} )}^{c 2} \times {( \hat{L} )}^{c 3} \times {( \hat{t} )}^{c 4} ... \times {π}_{m}^{cm} ) ,$$

and
c1>c2>c3>c4>cm, wherein terms where m>4 are optionally present.

According to a preferred aspect:
the zone is the RLL zone,

$$Eq . 1 is : {π}_{0 , zone} = {c}_{0} ( {( F P F )}^{c 1} \times {( Vent )}^{c 2} \times {( {V}_{branch , normal} )}^{c 3} \times {( \frac{Rate \cdot {( {t}_{inj} )}^{2}}{T {V}_{inh}} )}^{c 4} ... \times {π}_{m}^{c m} ) ,$$

and
c1>c2>c3>c4>cm, wherein terms where m>4 are optionally present.
   or
the zone is the RUL zone,

$$Eq . 1 is : {π}_{0 , zone} = {c}_{0} ( {( F P F )}^{c 1} \times {( Vent )}^{c 2} \times {( \frac{{Q}_{max}}{{Q}_{inh}} )}^{c 3} \times {( {V}_{branch , normal} )}^{c 4} ... \times {π}_{m}^{cm} ) ,$$

and
c1>c2>c3>c4>cm, wherein terms where m>4 are optionally present.
   or
the zone is the LUL zone,

$$Eq . 1 is : {π}_{0 , zone} = {c}_{0} ( {( F P F )}^{c 1} \times {( \frac{{Q}_{max}}{{Q}_{i n h}} )}^{c 2} \times {( \frac{{L}_{c h}}{M M A D} )}^{c 3} \times {( \frac{{A}_{b r a n c h}}{{A}_{t r a c h e a}} )}^{c 4} ... \times {π}_{m}^{c m} ) ,$$

and
c1>c2>c3>c4>cm, wherein terms where m>4 are optionally present.
   or
the zone is the RML zone,

$$Eq . 1 is : {π}_{0 , zone} = {c}_{0} ( {( F P F )}^{c 1} \times {( Vent )}^{c 2} \times {( Re )}^{c 3} \times {( \frac{{A}_{b r a n c h}}{{A}_{t r a c h e a}} )}^{c 4} ... \times {π}_{m}^{c m} ) ,$$

and
c1>c2>c3>c4>cm, wherein terms where m>4 are optionally present.
   or
the zone is the ET zone,

$$Eq . 1 is : {π}_{0 , zone} = {c}_{0} ( {( F P F )}^{c 1} \times {( \frac{{Q}_{max}}{{Q}_{i n h}} )}^{c 2} \times {( L N Param )}^{c 3} \times {( \frac{{L}_{c h}}{M M A D} )}^{c 4} ... \times {π}_{m}^{c m} ) ,$$

and
c1>c2>c3>c4>cm, wherein terms where m>4 are optionally present.
   or
the zone is the TL zone,

$$Eq . 1 is : {π}_{0 , zone} = {c}_{0} ( {( F P F )}^{c 1} \times {( \frac{{Q}_{max}}{{Q}_{inh}} )}^{c 2} \times {( {V}_{branch , normal} )}^{c 3} \times {( \frac{{L}_{c h}}{M M A D} )}^{c 4} ... \times {π}_{m}^{cm} ) ,$$

and
c1>c2>c3>c4>cm, wherein terms where m>4 are optionally present.

Also provided herein is a method for determining airway deposition of an inhaled substance in a lung of a subject, comprising:
receiving a patient dataset of the subject comprising a parameter set of two or more parameters and corresponding values,
determining, from the patient dataset, the airway deposition of the inhaled substance in the lung of the subject, wherein the parameter set comprises at least two parameters from Table B.

The parameter set may comprise:
at least one parameter from an imaging subgroup of the library of parameters of Table B,
at least one parameter from an inhaler subgroup library of parameters of Table B, and
at least one parameter from a flow subgroup library of parameters of Table B, and
at least one parameter from a physical properties and constants library of parameters of Table B.

The parameter set may comprise:
at least one of IM1, IM2, IM3, IM4 and IM6 from the imaging subgroup of Table B,
at least one of IN1, IN2 and IN5 from the inhaler subgroup of Table B,
at least one of F1, F2, and F4 from the flow subgroup of Table B, and
at least one of PC1, PC2 and PC3 of the physical properties and constants of Table B.

The determining preferably comprises:
applying the parameter set to a dimensionless correlation,
determining, from the patient dataset applied to the dimensionless correlation, the airway deposition of the inhaled substance in the lung of the subject,

Preferably:
a first component of the dimensionless correlation (e.g. y-axis) is a dimensionless number (π₀) representative of the airway deposition, and
a second component of the dimensionless correlation (e.g. x-axis) is a dimensionless group comprising one or more dimensionless numbers, wherein:
   the one or more dimensionless numbers are different from the dimensionless number of the first component, and
   the one or more dimensionless numbers are determined from the parameters in the parameter set.

At least some, preferably all of the dimensionless numbers in the second component of the dimensionless correlation may be determined using ratios and/or using Buckingham Pi theorem.

Exponent(s) of the dimensionless numbers of the dimensionless correlation may be determined using regression analysis and multiple patient record datasets, wherein patient record dataset comprises a record value for deposition, the parameters of the parameter set, and record value(s) of each parameter in the parameter set.

Preferably, the dimensionless correlation is determined according to a specific airway zone, and
the zone is an intrathoracic (IT) zone, the parameter set comprises the parameters of PPG6 in Table A, and the dimensionless numbers comprise the dimensionless numbers of PPG6 in Table A, or
the zone is an extrathoracic (ET) zone, the parameter set comprises the parameters of PPG7 in Table A, and the dimensionless numbers comprise the dimensionless numbers of PPG7 in Table A, or
the zone is a distal (DI) zone, the parameter set comprises the parameters of PPG8 in Table A, and the dimensionless numbers comprise the dimensionless numbers of PPG8 in Table A, or
the zone is a peripheral (PR), the parameter set comprises the parameters of PPG9 in Table A, and the dimensionless numbers comprise the dimensionless numbers of PPG9 in Table A, or
the zone is a total lobe (TL), the parameter set comprises the parameters of PPG10 in Table A, and the dimensionless numbers comprise the dimensionless numbers of PPG10 in Table A.

Preferably, the dimensionless correlation is determined according to a specific airway zone, and
the zone is a right upper lobe (RUL), the parameter set comprises the parameters of PPG11 in Table A, and the dimensionless numbers comprise the dimensionless numbers of PPG11 in Table A, or
the zone is a right middle lobe (RML), the parameter set comprises the parameters of PPG12 in Table A, and the dimensionless numbers comprise the dimensionless numbers of PPG12 in Table A, or
the zone is a right lower lobe (RLL), the parameter set comprises the parameters of PPG13 in Table A, and the dimensionless numbers comprise the dimensionless numbers of PPG13 in Table A, or
the zone is a left upper lobe (LUL), the parameter set comprises the parameters of PPG14 in Table A, and the dimensionless numbers comprise the dimensionless numbers of PPG14 in Table A, or
the zone is a lower left lobe (LLL), the parameter set comprises the parameters of PPG15 in Table A, and the dimensionless numbers comprise the dimensionless numbers of PPG15 in Table A.

Further provided herein is a computing device or system configured for performing the method according as described herein.

Further provided herein is a computer program or computer program product having instructions which when executed by a computing device or system cause the computing device or system to perform a method according as described herein.

### FIGURE LEGENDS

FIG. 1 graph showing multiple patient record datasets (training data) (black dots), a best fit line (dimensionless correlation) (dotted line) of the multiple patient record datasets, and test data (triangles) for the intrathoracic (IT) zone.

FIG. 2 graph showing multiple patient record datasets (training data) (black dots), a best fit line (dimensionless correlation) (dotted line) of the multiple patient record datasets, and test data (triangles) for the distal (DI) zone.

FIG. 3 graph showing multiple patient record datasets (training data) (black dots), a best fit line (dimensionless correlation) (dotted line) of the multiple patient record datasets, and test data (triangles) for the peripheral (PR) zone.

FIG. 4 graph showing multiple patient record datasets (training data) (black dots), a best fit line (dimensionless correlation) (dotted line) of the multiple patient record datasets, and test data (triangles) for the total lobes (TL) zone.

FIG. 5 graph showing multiple patient record datasets (training data) (black dots), a best fit line (dimensionless correlation) (dotted line) of the multiple patient record datasets, and test data (triangles) for the right upper lung (RUL) zone.

FIG. 6 graph showing multiple patient record datasets (training data) (black dots), a best fit line (dimensionless correlation) (dotted line) of the multiple patient record datasets, and test data (triangles) for the right middle lung (RML) zone.

FIG. 7 graph showing multiple patient record datasets (training data) (black dots), a best fit line (dimensionless correlation) (dotted line) of the multiple patient record datasets, and test data (triangles) for the right left lung (RLL) zone.

FIG. 8 graph showing multiple patient record datasets (training data) (black dots), a best fit line (dimensionless correlation) (dotted line) of the multiple patient record datasets, and test data (triangles) for the left upper lung (LUL) zone.

FIG. 9 graph showing multiple patient record datasets (training data) (black dots), a best fit line (dimensionless correlation) (dotted line) of the multiple patient record datasets, and test data (triangles) for the left lower lung (LLL) zone.

FIG. 10 graph showing calculation speed of the present method, dimensionless correlation (known as Rapid Deposition Analysis (RDA)), compared with a method of the art, computational fluid dynamics (CFD). Note scale is logarithmic.

FIG. 11 graph showing, for 18 test points (18 patients), a deposition average and standard deviation per zone obtained by NDA and CFD. The extremes of the boxes represent the quartiles (lower quartile is where 25% of data are less than that value, and upper quartile is where 25% of data is greater than that value). The horizontal lines gives the median, and the triangle give the mean value. The whiskers extend to the most extreme data point which is no more than 1.5 times the interquartile range from the box. All data points outside this range are visualized as individual points. The Mean and Standard Deviation are also given on the x-axis of each plot.

FIG. 12 graph of intercorrelations for deposition in the RML, as a function of different quantities of π's in Eq. 1a in Example 1.

FIG. 13 graph shows averaged results of depositions calculated using present method (RDA) when the quantity of π's in Eq. 1 was 4, compared with using CFD.

FIG. 14 graph shows averaged results of depositions calculated using present method (RDA) when the quantity of π's in Eq. 1 was 20, compared with using CFD.

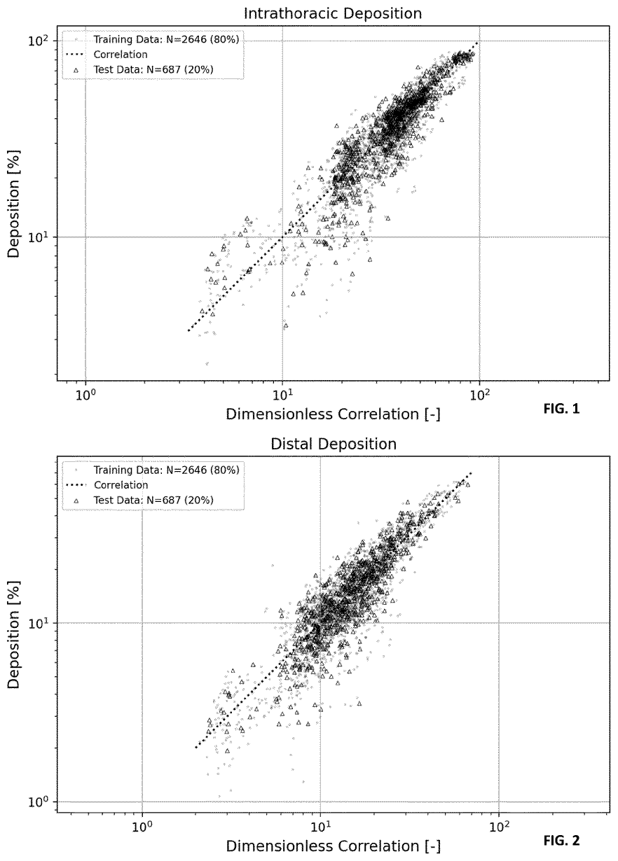

FIGS. 1 to 14 are inhaler-non-specific (all inhaler types together).

### DETAILED DESCRIPTION OF INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art.

For example, in the appended claims, any of the claimed embodiments can be used in any combination.

In the present description of the invention, reference is made to the accompanying drawings that form a part hereof, and in which are shown by way of illustration only of specific embodiments in which the invention may be practiced. Parenthesized or emboldened reference numerals affixed to respective elements merely exemplify the elements by way of example, with which it is not intended to limit the respective elements. Unless otherwise indicated, all figures and drawings in this document are not to scale and are chosen for the purpose of illustrating different embodiments of the invention. In particular the dimensions of the various components are depicted in illustrative terms only, and no relationship between the dimensions of the various components should be inferred from the drawings, unless so indicated.

It is to be understood that other embodiments may be utilised and structural or logical changes may be made without departing from the scope of the present invention. The following detailed description, therefore, is not to be taken in a limiting sense, and the scope of the present invention is defined by the appended claims.

Provided herein is a method for determining airway deposition of an inhaled substance in a lung of a subject, comprising:
receiving a patient dataset of the subject comprising a parameter set of two or more parameters and corresponding values,
determining, from the patient dataset, the airway deposition of the inhaled substance in the lung of the subject,
   wherein the parameter set comprises at least two parameters from Table B.

The inventors have found that parameters of subject can be arranged into different dimensionless numbers of a dimensionless correlation particular to a zone of the subject, and the dimensionless correlation is predictive of deposition within the zone. Depending on the zone, different dimensionless correlations may be formed. It has not been previously understood that dimensionless numbers are predictive of deposition in different locations of the lungs i.e. different zones. By employing a correlation in place of Computational Fluid Dynamics (CFD) to predict deposition, the speed of calculating deposition is significantly enhanced.

The airway deposition may be determined for one airway zone (aka “zone” herein) out of a plurality of zones. In other words, the determination is specific to one zone. Multiple determinations may be performed, each for a different zone.

The plurality of zones comprise the following: total lobes (TL), intrathoracic (IT), extrathoracic (ET), distal (DI), peripheral (PR), right upper lobe (RUL), right middle lobe (RML), right lower lobe (RLL), left upper lobe (LUL), left lower lobe (LLL). The zones and the airways they include, are known in the art.

As understood in the art a lobe is a segment of a lung. The right lung has three major lobes (RUL, RML, RLL); the left lung, which is slightly smaller than the right lung because of the asymmetrical placement of the heart, has two lobes (LUL and LLL).

As understood in the art, total lobes (TL) means (RUL+RML+RLL+LUL+LLL); intrathoracic (IT) refers to airways below (inferior to) the top of the trachea (includes central, distal and peripheral); extrathoracic (ET) refers to airways above (superior to) the top of the trachea. Central (CL) refers to airways that from the top of the trachea to main bronchus visible in a CT scan.

Distal (DI) refers to airways that branch from the main bronchus visible in a CT scan. These are typically airways with a diameter above 1 to 2 mm. These airways reach out as far as a 7-10th branching generation. Peripheral (PR) refers to airways that cannot be distinguished on a CT scan. In terms of particle deposition, a particle is deposited in the peripheral (PR) airways when this particle exits a model of the distal (DI) airways. The upper airway as used herein, refers to the combination of the oral cavity, pharynx, and larynx.

As understood in the art an airway is a conduit for air between a mouth/nose and lung of the subject, having a lumen. Airways present in a zone may have a surface area, which refers to a total surface of airway lumen (inner wall) within the zone. Airways present in a zone may have a volume, which refers to a total volume of airway lumens within the zone.

According to one aspect, the airway deposition is determined for one zone in an order of preference (most preferred to less preferred) that is IT (PPG6)>PR (PPG9)>DI (PPG8). According to one aspect, the airway deposition is determined for one or at least airway zone that is IT (PPG6).

Airway deposition (also known as “deposition”, or “De”, or “no” herein) refers to an amount of inhaled substance that is deposited in the zone of the subject. It is expressed in terms of a fraction, ratio or percentage of the delivered dose, i.e. dose (by mass) deposited in zone cf the total dose (by mass) inhaled by a subject. It is a dimensionless number (i.e. without units).

The inhaled substance is a liquid aerosol or powered aerosol. It is usually administered using an inhaler. The inhaled substance typically contains an active agent. Typical types of inhaler include a dry powder inhaler (DPI), a nebulizer, and a meter dose (MDI) inhaler. In a dry powder inhaler (DPI), the inhaled substance is a dry powder; inhalation by the patient a primary force driving movement of the dry powder. In a nebulizer, the inhaled substance is a liquid, driven by inhalation; inhalation by the patient a primary force driving movement of the liquid. In a meter dose (MDI) inhaler, the inhaled substance is a liquid, a part of the force driving movement of the liquid is pressurised propellant. Examples of inhaled substances include Insulin, salbutamol, Promixin, Tobramycin. Examples of commercially available products inhalers containing one or more active agents include Ellipta DPI (Fluticasone furoate+Vilanterol); Seretide MDI (Fluticasone furoate+Salmeterol); Symbicort Turbohaler DPI (Budesonide+Formoterol Fumarate); Quinsair Zirela Nebulizer.

The parameter set comprises parameters that are deterministic for the airway deposition, in particular, in a zone.

The parameter set comprises at least two parameters from the library of parameters of Table B. The parameter set preferably comprises at least 9, preferably 9 to 17 of the parameters from Table B. Each parameter in Table B has a value that is related to the patient (test or record) inhaling the substance. Some parameters are related to physiology of the patient (e.g. imaging subgroup of Table B). Some parameters are related to the inhaler and aerosol used by the patient (e.g. inhaler subgroup of Table B). Some parameters are related to air or substance flow during inhalation by the patient (e.g. flow subgroup of Table B). Some parameters are physical properties and constants (e.g. Physical properties and constants of Table B). It is understood that the parameter set may contain, in addition to some of the parameters from the library of parameters of Table B, or in addition to the parameters of one PPG in Table A, one or more parameters not listed in Table A or B.

Preferably at least two parameters from the library of parameters of Table B form at least one dimensionless number. Preferably the parameters from the library of parameters of Table B form at least 4, between 4 and 20, preferably between 4 and 9, more preferably 4, 5 or 6 dimensionless numbers. A dimensionless number (DN/π) is an equation term that has no dimensions i.e. no units. One way to create a dimensionless number is to divide two parameters with the same primary dimensions. For instance, a ratio of IM1 and IM2 in Table B forms dimensionless number DN6 in Table C. For instance, a ratio of IM4 and IM6 in Table B forms dimensionless number DN3 in Table C. Another way to create dimensionless numbers is using Buckingham Pi Theorem described elsewhere herein.

The parameter set may comprise:
at least one parameter from an imaging subgroup of the library of parameters of Table B,
at least one parameter from an inhaler subgroup library of parameters of Table B, and
at least one parameter from a flow subgroup library of parameters of Table B, and
at least one parameter from a physical properties and constants library of parameters of Table B.

Parameters of the imaging subgroup (IM1 to IM11) are set out in Table B. Parameters of the imaging subgroup are patient-specific. Parameters of the inhaler subgroup (IN1 to IN6) are set out in Table B. Parameters of the flow subgroup (F1 to F10) are set out in Table B. Parameters of the physical properties and constants subgroup (PC1 to PC12) are set out in Table B.

The parameter set may comprise:
at least one of IM1, IM2, IM3, IM4 and IM6 from the imaging subgroup,
at least one of IN1, IN2 and IN5 from the inhaler subgroup,
at least one of F1, F2, and F4 from the flow subgroup, and
at least one of PC1, PC2 and PC3 of the physical properties and constants

The parameter set may comprise:
at least IM1, IM2, IM3, IM4 and IM6 from the imaging subgroup,
at least IN1, IN2 and IN5 from the inhaler subgroup,
at least F1, F2, and F4 from the flow subgroup, and
at least PC1, PC2 and PC3 of the physical properties and constants

The parameter set may comprise at least two parameters from one of the primary parameter groups PPG1 to PPG15 of Table A.

The parameter set preferably comprises at least two parameters, preferably 10 to 17 of the parameters from one of the primary parameter groups PPG2 to PPG5 in Table A. The parameter set preferably forms at least 4, between 4 and 20, preferably between 4 and 9, more preferably 4, 5 or 6 dimensionless numbers, in the corresponding primary parameter group of Table A. For instance, if the parameter set is chosen from PPG3 in Table A, and comprises parameters A_{trachea}, A_{min,ua} and A_{branch}, then they can form two of the dimenionless numbers of PPG3 in Table A, namely A_{min,ua}/A_{trachea}, and A_{branch}/A_{trachea}.

The parameter set preferably comprises at least two parameters, preferably all of the parameters from one of the primary parameter groups PPG6 to PPG15 of Table A, depending on the zone.

The parameter set preferably comprise at least the parameters of one of the primary parameter groups PPG6 to PPG15 of Table A1, depending on the zone. The parameter set preferably forms at least the 4 dimensionless in the corresponding primary parameter group of Table A1. The zone may be one or more of IT, PR, DI, RLL, LLL, RUL, LUL, RML, ET or TL.

According to one aspect, provided is a method for airway deposition of an inhaled substance in multiple zones of a lung of a subject, using the methods described herein applied per zone of the multiple zones. The zones of the multiple zones may be two or more of IT, PR, DI, RLL, LLL, RUL, LUL, RML, ET or TL. Preferably, the zones of the multiple zones comprise IT and optionally one or more of PR, DI, RLL, LLL, RUL, LUL, RML, ET, TL. Preferably, the zones of the multiple zones comprise IT and PR, and optionally one or more of DI, RLL, LLL, RUL, LUL, RML, ET, TL. Even more preferably, the zones of the multiple zones comprise IT, PR and DI, and optionally one or more of RLL, LLL, RUL, LUL, RML, ET, TL.

The parameter set per zone preferably comprises at least the parameters of the primary parameter groups PPG6 to PPG15 of Table A1, depending on the zone. The parameter set preferably per zone preferably forms at least the 4 dimensionless in the corresponding primary parameter group of Table A1.

It is understood that parameter sets may be determined using dimensional analysis (also called non-dimensional analysis (NDA)). Typically, one parameter set per zone is determined. The parameter set may be refined and hence change over time as additional patient records are used to determine the parameter set (see later below). Alternatively, once the parameter set is determined for a zone, it may remain unchanged.

Different primary parameter groups are shown in Table A. Each row of Table A contains one primary parameter group.

Primary parameter group 1 (PPG1) in Table A contains the parameters of the parameter library of Table C; each parameter has been found to be important for determining deposition in one or more zones.

Primary parameter group 2 (PPG2) in Table A contains parameters found to be important for determining deposition in one or more zones. The parameters are those found most frequently as determinants of airway deposition in different zones, and they are parameters which are less frequent but in a dimensionless number with a higher weighting (exponent). Also shown in Table A are the dimensionless numbers formed from the parameters of PPG2.

Primary parameter group 3 (PPG3) in Table A contains parameters found experimentally to be the most deterministic for airway deposition. The parameters are those found most frequently as determinants of airway deposition in different zones, and they are parameters which are less frequent but in a dimensionless number with a higher weighting (exponent). PPG3 corresponds to the Example herein. Also shown in Table A are the dimensionless numbers formed from the parameters of PPG3.

Primary parameter group 4 (PPG4) in Table A contains parameters found experimentally to be the most deterministic for airway deposition. The parameters are those found most frequently as determinants of airway deposition in almost all of different zones. Also shown in Table A are the dimensionless numbers formed from the parameters of PPG4.

Primary parameter group 5 (PPG5) in Table A contains parameters found experimentally to be the most deterministic for airway deposition. The parameters are those found most frequently as determinants of airway deposition, and are present in most of the different zones. Also shown in Table A are the dimensionless numbers formed from the parameters of PPG5.

Primary parameter group 6 (PPG6) in Table A or A1 contains parameters found experimentally to be the most deterministic for airway deposition in the Intrathoracic (IT) zone. Also shown in Table A or A1 are the dimensionless numbers formed from the parameters of PPG6.

Primary parameter group 7 (PPG7) in Table A or A1 contains parameters found experimentally to be the most deterministic for airway deposition in the Extrathoracic (ET) zone. Also shown in Table A or A1 are the dimensionless numbers formed from the parameters of PPG7.

Primary parameter group 8 (PPG8) in Table A or A1 contains parameters found experimentally to be the most deterministic for airway deposition in the Distal (DI) zone.

Also shown in Table A or A1 are the dimensionless numbers formed from the parameters of PPG8.

Primary parameter group 9 (PPG9) in Table A or A1 contains parameters found experimentally to be the most deterministic for airway deposition in the Peripheral (PR) zone. Also shown in Table A or A1 are the dimensionless numbers formed from the parameters of PPG9.

Primary parameter group 10 (PPG10) in Table A or A1 contains parameters found experimentally to be the most deterministic for airway deposition in the Total Lobe (TL) zone. Also shown in Table A or A1 are the dimensionless numbers formed from the parameters of PPG10.

Primary parameter group 11 (PPG11) in Table A or A1 contains parameters found experimentally to be the most deterministic for airway deposition in the Right upper lobe (RUL) zone. Also shown in Table A or A1 are the dimensionless numbers formed from the parameters of PPG11.

Primary parameter group 12 (PPG12) in Table A or A1 contains parameters found experimentally to be the most deterministic for airway deposition in the Right middle lobe (RML) zone. Also shown in Table A or A1 are the dimensionless numbers formed from the parameters of PPG12.

Primary parameter group 13 (PPG13) in Table A or A1 contains parameters found experimentally to be the most deterministic for airway deposition in the Right lower lobe (RLL) zone. Also shown in Table A or A1 are the dimensionless numbers formed from the parameters of PPG13.

Primary parameter group 14 (PPG14) in Table A or A1 contains parameters found experimentally to be the most deterministic for airway deposition in the Left upper lobe (LUL) zone. Also shown in Table A or A1 are the dimensionless numbers formed from the parameters of PPG14.

Primary parameter group 15 (PPG15) in Table A or A1 contains parameters found experimentally to be the most deterministic for airway deposition in the Left lower lobe (LLL) zone. Also shown in Table A or A1 are the dimensionless numbers formed from the parameters of PPG15.

The patient dataset of the subject comprises:
the parameters of the parameter set, and
value(s) of each parameter in the parameter set.

The airway deposition of the inhaled substance in the lung of the subject is determined from the patient dataset applied to a dimensionless correlation. The dimensionless correlation is a mathematical correlation between airway deposition and parameters in the parameter set. The dimensionless correlation is preferably particular to a zone. For instance, dimensionless correlation_{RUL} may be different from a dimensionless correlation_{intrathoracic}.

In particular, the airway deposition of the inhaled substance in the lung of the subject is determined from the dimensionless correlation, wherein:
a first component of the correlation (e.g. y-axis) is a dimensionless number (π₀) representative of the airway deposition, and
a second component of the correlation (e.g. x-axis) is a dimensionless group comprising one or more dimensionless numbers. The one or more dimensionless numbers are different from the dimensionless number of the first component. The one or more dimensionless numbers are formed the parameters in the parameter set.

The dimensionless correlation may be expressed as:

$$π 0 = {c}_{0} ( {π}_{1}^{c 1} \times {π}_{2}^{c 2} \times {π}_{3}^{c 3} \times ... \times {π}_{m}^{cm} ) \qquad [ Eq . 1 ]$$

where
deposition in a zone is represented as a dimensionless number (π₀) (first component),
c₀ (π₁^{c1}×π₂^{c2}×π₃^{c3}× . . . ×πₘ^{cm}) is the dimensionless group (second component),
each π of π_{1 to m} is a dimensionless number for the zone,
each π^{c} of π₁^{c1} to πₘ^{cm} is a dimensionless term,
each c of c1 to cm is an exponent of the dimensionless number (“exponents” herein),
c₀ is a multiplier of the correlation,
m is at least 4, between 4 and 20, preferably between 4 and 9, more preferably 4, 5 or 6,
   each of π₀ for the zone may be represented as π_{0,zone}.

The dimensionless correlation hence comprises a dimensionless group of one or more dimensionless numbers, each dimensionless number formed from one or more of the parameters in the set of parameters.

The dimensionless group may comprise one or more dimensionless numbers from the list in Table C. The dimensionless group may comprise at least one dimensionless number from one of the primary parameter groups (PPG1 to PPG15) (Table A). The dimensionless group preferably comprises at least two dimensionless numbers, preferably 6 to 9, of the dimensionless number from one of the primary parameter groups PPG3 to PPG5) (Table A). The dimensionless group preferably comprises at least two dimensionless numbers, preferably all of the dimensionless numbers from one of the primary parameter groups PPG6 to PPG15) (Table A), depending on the zone.

It is understood that dimensionless numbers in the dimensionless group starting from any set of parameters, may be determined using Buckingham Pi Theorem, known in the art, for instance, from Buckingham, Nature 96.2406 (1915): 396-397, or Gibbings, John Cecil. Dimensional analysis. Springer Science & Business Media, 2011.

Each c of c1 to cm, and the multiplier c₀ may be determined using regression analysis and the multiple patient record datasets.

The processes of determining the dimensionless correlation, including one or more steps of determining primary dimension of a parameter, forming dimensionless numbers, forming a dimensionless group, solving multiplier and exponents of the dimensionless correlation, are known as dimensional analysis or non-dimensional analysis (NDA). As background information, a dimensionless number (DN/π) is an equation term that has no dimensions i.e. no units. Preferably, the number of dimensionless numbers in the dimensionless group is at least one or two. The number of dimensionless groups in the second component is one. Each dimensionless number (DN/π) in the dimensionless group contains one or more parameters; a non-exhaustive list of parameters is provided in the library of Table B. The one or more dimensionless numbers (π₁, π₂, π₃ . . . πₘ) forming the dimensionless group is determined by Buckingham Pi Theorem. In Table A and C, for instance, the dimensionless number were determined using Buckingham Pi Theorem. Where other parameters have been determined not listed in Table B, Buckingham Pi Theorem may be used to determine dimensionless numbers therefrom, optionally in combination with one or more parameters listed in Table B. The values of c₀, c1 to cm are determined by solving a nonlinear least-squares equation.

The inventors have found that the second component of the dimensionless correlation expressed as one or more dimensionless numbers (DN/r), allows the problem of airway deposition for a zone to be reduced to a two-component correlation.

The dimensionless numbers for each primary parameter group have been determined in Table A. Where a new primary parameter group is formed containing parameters not listed in parameter library of Table B, corresponding dimensionless numbers may be determined by taking ratios or using the Buckingham Pi theorem.

A typical application of the Buckingham Pi Theorem is as follows:

Form the primary parameter group. The primary parameter group may, for example, comprising at least some, or all of the parameters from the parameter library of Table B and optionally one or more additional parameters not indicated in Table B. Alternatively, the primary parameter group may comprise at least some, preferably all of the parameters of one of the primary parameter groups of Table A or A1 and optionally one or more additional parameters not indicated in Table B. In an example, the primary parameter group may be PPG3 in Table A.

For each parameter in the primary parameter group, its primary dimension(s) is determined i.e. M (mass), L (length) and/or t (time) and/or Θ (temperature) (see column Primary Dimension in Table B).

The number (j) of primary dimensions in the primary parameter group is determined (usually 3 (M,L,T) or 4 (M,L,T, Θ) primary dimensions) e.g. based on Table A, PPG3: j=4.

The number (n) of parameters in the deposition and primary parameter group is determined (e.g. based on Table A, PPG3: n=34).

The minimum number (k) of dimensionless numbers (k=n−j) in the deposition and primary parameter group is determined (e.g. based on Table A, PPG3: k=34−4=30 (at least). Select j repeating variables from the primary parameter group. None of the repeating variables may be dimensionless (e.g. may not choose Nₒᵤₜₗₑₜ, FPF, GSD, α). No two repeating variables may have an identical overall dimension. (e.g. may not choose V_{FRC} or TVᵢₙₕ (both L³)). Two repeating variables may share a common dimension. It is generally advantageous to choose parameters in which the primary dimensions relate to mass, geometry and kinematics. By geometry, it is meant the parameters related to the structure of a zone and is usually one of the imaging subgroups of Table B. By kinetics, it is meant the parameters that cause motions such as velocity and air flow. (e.g. based on Table A, PPG3: j=4);

Constructing dimensionless numbers (π_{1 to} πₖ₋₁). To construct a dimensionless number (e.g. π₁), form a holding expression comprising the repeating variables and one “remaining parameter” from the primary parameter group which is not one of the repeating variables. Rearrange the parameters, expressed as primary dimensions, in the holding expression with an aim of forming one dimensionless numbers and optionally removing parameters.

The process is repeated for the next dimensionless number (e.g. π₂) in the primary parameter group. The holding expression contains the same repeating variables and one “remaining parameter” is one not used before. (e.g. based on Table A, PPG3: the dimensionless numbers (DN) shown from PPG3 have derived from parameters of PPG3) At the end of the application of the Buckingham Pi Theorem, there are at least k dimensionless numbers (π₀ and π_{1 to} πₖ₋₁), wherein each π in π_{1 to} πₖ₋₁ is defined in terms parameters in the primary parameter group.

A patient record dataset comprises:
a record value for deposition,
the parameters of the parameter set, and
record value(s) of each parameter in the parameter set.

A record value is a value that has been previously determined for a patient. Multiple record values are obtained from multiple patient record datasets. The multiple patient record dataset are used to solve the multiplier and exponents in the dimensionless correlation.

The record value for deposition contains an indication of the zone of deposition. A record value for deposition may be determined from medical imaging (e.g. Single photon emission computed tomography (SPECT)) and gamma scintigraphy using a radiolabelled aerosol. These methods are known in the art (e.g. Conway, Joy. (2012). Lung imaging—two dimensional gamma scintigraphy, SPECT, CT and PET. Advanced drug delivery reviews. 64. 357-68. 10.1016/j.addr.2012.01.013.). Alternatively, or in addition a record value for deposition may be determined from medical imaging and Computational Fluid Dynamics (CFD). These methods are known in the art (e.g. De Backer et al., J Aerosol Med Pulm Drug Deliv. 2010 June; 23(3):137-48; Usmani, Omar S., et al. “Predicting lung deposition of extrafine inhaled corticosteroid-containing fixed combinations in patients with chronic obstructive pulmonary disease using functional respiratory imaging: An in silico study.” Journal of aerosol medicine and pulmonary drug delivery 34.3 (2021): 204-211). It is understood that a patient record dataset may contain values for some or all the parameters in the parameter library (Table B). A portion of the parameters available in a patient record dataset is used to populate the parameter set. Typically, if a patient record dataset is lacking one or more parameter values for parameters of the parameter set, patient record dataset is not used in the regression analysis.

The value(s) of a parameter for a patient (test value) is used for determining deposition. The value(s) of parameter in the patient record (record value) is used for determining the dimensionless correlation, in particular, values of the multiplier and the value of each exponent in Eq. 1.

The value(s) of a parameter for a patient (test or record) that is an imaging parameter (e.g. IM to IM11 in Table B) may be determined from medical imaging (e.g. sliced CT scan or Volumetric CT scan). These methods are known in the art. See for instance “Machine learning algorithms utilizing quantitative CT features may predict eventual onset of bronchiolitis obliterans syndrome after lung transplantation.” Academic radiology 25.9 (2018): 1201-1212; De Backer, Wilfried, et al. “Functional respiratory imaging assessment of glycopyrrolate and formoterol fumarate metered dose inhalers formulated using co-suspension delivery technology in patients with COPD.” Therapeutic advances in respiratory disease 14 (2020): 1753466620916990.

The value(s) of a parameter for a patient (test or record) that is an inhaler parameter (e.g. IN1 to IN6 in Table B) may be determined from manufacturer data of the inhaler or Next Generation Impactor (NGI) technique. The Next Generation Impactor™, or NGI™, has a cascade impactor to categorize aerosols of an inhaler based on their sizes. The output of an NGI™, is typically a list of absolute mass of the aerosols deposited in each stage the impactor with the diameter range related to that stage. Based on these two, a size histogram may be created for the emitted aerosols from an inhaler.

The value(s) of a parameter for a patient (test or record) that is a flow parameter (e.g. F1 to F10 in Table B) may be determined from, for instance, from a manufacturer's recommendation of the way to inhale/breath while using an inhaler, or the way a patient actually inhales/breathes while using an inhaler. It is dependent on the inhaler used. The value(s) of a parameter for a patient (test or record) that is a physical property or constant (e.g. PC1 to PC12 in Table B). Typically these are values that may be set as constants. Constants can be obtained from Thermodynamic tables. However, it is within the scope of the present disclosure that one or more of the parameters can vary according to the environment of the patient e.g. room temperature.

For the dimensionless group in Eq. 1, value of the multiplier and the value of each exponent in Eq. 1 may be determined using regression analysis based on the multiple patient record datasets.

For example, where the dimensionless group in Eq. 1 comprises:
at least one dimensionless number from the list in Table C,
at least one dimensionless number from one of the primary parameter groups PPG1 to PPG2 (Table A),
at least two dimensionless numbers, preferably 6 to 9, of the dimensionless number from one of the primary parameter groups PPG3 to PPG5 (Table A),
at least two dimensionless numbers, preferably all of the dimensionless numbers from one of the primary parameter groups PPG6 to PPG15 (Table A), depending on the zone,
the at least four dimensionless number from one of the primary parameter groups PPG6 to PPG15 (Table A1),
or
any of the above in addition to one or more dimensionless numbers not listed in Table C,
   the multiplier and each exponent in Eq. 1 may be determined using regression analysis based on the multiple patient record datasets.

Regression analysis is known in the art. Generally speaking, the values of the multiplier and each exponent in Eq. 1 are adjusted (e.g. towards higher or lower values) iteratively, such that the value of the dimensionless group (when multiple patient record datasets are applied) approaches the value of the deposition (π₀) (in multiple patient record datasets). Typically, convergence to a solution may be reached within a certain number of iterations.

Various types of regression analysis are suitable for determining the values of the multiplier and exponents. A preferred type of regression analysis is non-linear least-squares (NLSQ) protocol. In particular, a Levenberg-Marquardt method or algorithm (J. J. More, “The Levenberg-Marquardt Algorithm: Implementation and Theory,” Numerical Analysis, ed. G. A. Watson, Lecture Notes in Mathematics 630, Springer Verlag, pp. 105-116, 1977) is most preferred. During or as a result of the regression analysis, one or more values of c1 to cm might tend to zero, thereby removing one or more dimensionless numbers.

In order to provide zone specificity to a dimensionless correlation, the multiple patient record datasets using in the regression analysis are pre-filtered to include only patient record datasets with deposition values particular to the zone of interest.

While the Buckingham Pi Theorem may create at least k−1 dimensionless numbers (a “full set”) in the dimensionless group from the primary parameter set, we have found that using above 4 dimensionless numbers or dimensionless terms does not lead to a significant gain in accuracy, but it does lead to an improvement. Preferably, the quantity of dimensionless numbers or dimensionless terms in the dimensionless group of the dimensionless correlation is preferably between 4 and 9, more preferably 4, 5, or 6. Accordingly, the number of coefficients is preferably between 4 and 9, more preferably 4, 5, or 6. Having a limited number of exponents leads to faster convergence in the process of finding values for the multiplier and exponents.

The dimensionless group may hence contain a reduced set of dimensionless numbers or terms (“reduced set”), selected from a full set of dimensionless numbers (“full set”). The reduced set preferably contains a quantity of the dimensionless numbers or terms equal to a term limit (m). The term limit (m) of the reduced set is preferably between 4 and 9, more preferably 4, 5, or 6.

The reduced set is determined using a remove and replace protocol. In the remove and replace protocol, the dimensionless group of the dimensionless correlation of Eq 1 is populated initially with a set of replaceable dimensionless terms or numbers (“replaceable set”) randomly selected from the full set, until a quantity (m) of dimensionless numbers or terms present in the dimensionless group is equal to or greater than 6, preferably between 6 and 12, more preferably between 8 and 10. In a first round, regression analysis, as described herein is performed on the dimensionless correlation in which the dimensionless group contains the replaceable set of dimensionless numbers or terms, and using the multiple patient record datasets. The regression analysis adjusts values for c₀, c1 to cm until there is convergence to a solution, and values for the multiplier c₀ and exponents c1 to cm are determined. An interclass correlation (ICC) is determined for the first round.

In a second round, a substitute quantity of dimensionless numbers or terms is randomly removed from the replaceable set of the first round, and the substitute quantity of different dimensionless numbers is randomly selected from the full set and added. The substitute quantity may be a value between 3 and 5. In the second round, the regression analysis adjusts values for c₀, c1 to cm until there is convergence to a solution, providing a second set of weight values for c₀, c1 to cm. The interclass correlation (ICC) is determined (typically above 0.9).

If the ICC improves in the second round compared with the first round, a third round continued with the replaceable set of dimensionless numbers or terms of the second round. If the ICC is worse in the second round compared with the first round, a third round continues with the first round. Future rounds proceed as for previous rounds, mutatis mutandis. The process is repeated until insignificant change in ICC. This typically happens after 500 to 1000 rounds.

Dimensionless terms of the replaceable set which are below a significance threshold value and removed, thereby forming the reduced set of dimensionless numbers or terms, typically with preferably between 4 and 9, more preferably 4, 5 or 6 dimensionless numbers or terms. For instance, if a contribution of a dimensionless term is below 0.05% to 0.2%, preferably 0.1% of a total value (p0, RML), then it is removed.

The full set of the dimensionless numbers or terms mentioned above (also known as pool of dimensionless numbers) may comprise the dimensionless numbers of Table C, preferably comprise the dimensionless numbers of Table A, PPG 2, more preferably comprise the dimensionless numbers of Table A, PPG 3, even more preferably comprise the dimensionless numbers of Table A, PPG 4, most preferably comprise the dimensionless numbers of Table A, PPG 5.

According to one aspect each of π₁ of π_{1 to} πₘ, each c of c1 to cm, and c₀ for the zone is determined by:
populating the dimensionless group of Eq. 1 with at least 9 dimensionless numbers (replaceable set) selected from the pool of dimensionless numbers,
performing multiple rounds regression analysis, wherein each round comprises:
   adjusting values for c₀, c1 to cm until there is convergence to a solution for deposition for the multiple patient record datasets,
   substituting a portion of the dimensionless numbers (e.g. 2 to 5) in the dimensionless group and with different dimensionless numbers selected from the pool of dimensionless numbers, and
repeating the adjusting and substituting until improvement in convergence (interclass correlation) is minimal or essentially none or tends to zero.

An exemplary reduced set of the dimensionless numbers per zone is set out in Table A and Table A1 (PPG6 to PPG15). The reduced set of the dimensionless numbers mentioned above preferably comprise the dimensionless numbers of Table A or A1 dependent on the zone, namely PPG6 (for IT zone deposition), PPG6 (for IT zone deposition), PPG7 (for ET zone deposition), PPG8 (for DI zone deposition), PPG9 (for PR zone deposition), PPG10 (for TL zone deposition), PPG11 (for RUL zone deposition), PPG12 (for RML zone deposition), PPG13 (for RLL zone deposition), PPG14 (for LUL zone deposition), PPG15 (for LLL zone deposition).

According to one aspect:
the zone is the IT zone,

$$Eq . 1 is : {π}_{0 , zone} = {c}_{0} ( F P {F}^{c 1} \times {( \frac{{Q}_{max}}{{Q}_{inh}} )}^{c 2} \times L N {Param}^{c 3} \times {( \frac{{L}_{c h}}{M M A D} )}^{c 4} ... \times {π}_{m}^{cm} ) ,$$

and
c1>c2>c3>c4>cm.

According to one aspect:
the zone is the IT zone,

$$Eq . 1 is : {π}_{0 , zone} = {c}_{0} ( F P {F}^{c 1} \times {( \frac{{Q}_{max}}{{Q}_{inh}} )}^{c 2} \times L N {Param}^{c 3} \times {( \frac{{L}_{c h}}{M M A D} )}^{c 4} ) ,$$

and
c1>c2>c3>c4>cm.

According to one aspect:
the zone is the PR zone,

$$Eq . 1 is : {π}_{0 , zone} = {c}_{0} ( {( F P F )}^{c 1} \times {( \frac{{Q}_{max}}{{Q}_{i n h}} )}^{c 2} \times {( Re )}^{c 3} \times {( β )}^{c 4} ... \times {π}_{m}^{c m} ) ,$$

and
c1>c2>c3>c4>cm.

According to one aspect:
the zone is the PR zone,

$$Eq . 1 is : {π}_{0 , zone} = {c}_{0} ( {( F P F )}^{c 1} \times {( \frac{{Q}_{max}}{{Q}_{i n h}} )}^{c 2} \times {( Re )}^{c 3} \times {( β )}^{c 4} ) ,$$

and
c1>c2>c3>c4.

According to one aspect:
the zone is the DI zone,

$$Eq . 1 is : {π}_{0 , zone} = {c}_{0} ( {( F P F )}^{c 1} \times {( s td L N )}^{c 2} \times {( \hat{L} )}^{c 3} \times {( \frac{{Q}_{max}}{{Q}_{i n h}} )}^{c 4} ... \times {π}_{m}^{c m} ) ,$$

and
c1>c2>c3>c4>cm.

According to one aspect:
the zone is the DI zone,

$$Eq . 1 is : {π}_{0 , zone} = {c}_{0} ( {( F P F )}^{c 1} \times {( s td L N )}^{c 2} \times {( \hat{L} )}^{c 3} \times {( \frac{{Q}_{max}}{{Q}_{i n h}} )}^{c 4} ) ,$$

and
c1>c2>c3>c4.

According to one aspect:
the zone is the RLL zone,

$$Eq . 1 is : {π}_{0 , zone} = {c}_{0} ( {( F P F , )}^{c 1} \times {( {V}_{branch , normal} )}^{c 2} \times {( \hat{L} )}^{c 3} \times {( \hat{t} )}^{c 4} ... \times {π}_{m}^{c m} ) ,$$

and
c1>c2>c3>c4>cm.

According to one aspect:
the zone is the RLL zone,

$$Eq . 1 is : {π}_{0 , zone} = {c}_{0} ( {( FPF , )}^{c 1} \times {( {V}_{branch , normal} )}^{c 2} \times {( \hat{L} )}^{c 3} \times {( \hat{t} )}^{c 4} ) ,$$

and
c1>c2>c3>c4>cm.

According to one aspect:
the zone is the LLL zone,

$$Eq . 1 is : {π}_{0 , zone} = {c}_{0} ( {( FPF )}^{c 1} \times {( Vent )}^{c 2} \times {( {V}_{branch , normal} )}^{c 3} \times {( \frac{Rate \cdot {( {t}_{inj} )}^{2}}{{TV}_{inh}} )}^{c 4} … \times {π}_{m}^{cm} ) ,$$

c1>c2>c3>c4>cm.

According to one aspect:
the zone is the LLL zone,

$$Eq . 1 is : {π}_{0 , zone} = {c}_{0} ( {( FPF )}^{c 1} \times {( Vent )}^{c 2} \times {( {V}_{branch , normal} )}^{c 3} \times {( \frac{Rate \cdot {( {t}_{inj} )}^{2}}{{TV}_{inh}} )}^{c 4} ,$$

and
c1>c2>c3>c4.

According to one aspect:
the zone is the RUL zone,

$$Eq . 1 is : {π}_{0 , zone} = {c}_{0} ( {( FPF )}^{c 1} \times {( Vent )}^{c 2} \times {( \frac{{Q}_{max}}{{Q}_{inh}} )}^{c 3} \times {( {V}_{branch , normal} )}^{c 4} … \times {π}_{m}^{cm} ) ,$$

and
c1>c2>c3>c4>cm.

According to one aspect:
the zone is the RUL zone,

$$Eq . 1 is : {π}_{0 , zone} = {c}_{0} ( {( FPF )}^{c 1} \times {( Vent )}^{c 2} \times {( \frac{{Q}_{max}}{{Q}_{inh}} )}^{c 3} \times {( {V}_{branch , normal} )}^{c 4} ) ,$$

and
c1>c2>c3>c4.

According to one aspect:
the zone is the LUL zone,

$$Eq . 1 is : {π}_{0 , zone} = {c}_{0} ( {( FPF )}^{c 1} \times {( \frac{{Q}_{max}}{{Q}_{inh}} )}^{c 2} \times {( \frac{{L}_{ch}}{MMAD} )}^{c 3} \times {( \frac{{A}_{branch}}{{A}_{trachea}} )}^{c 4} … \times {π}_{m}^{cm} ) ,$$

c1>c2>c3>c4.

According to one aspect:
the zone is the LUL zone,

$$Eq . 1 is : {π}_{0 , zone} = {c}_{0} ( {( FPF )}^{c 1} \times {( \frac{{Q}_{max}}{{Q}_{inh}} )}^{c 2} \times {( \frac{{L}_{ch}}{MMAD} )}^{c 3} \times {( \frac{{A}_{branch}}{{A}_{trachea}} )}^{c 4} ) ,$$

and
c1>c2>c3>c4>cm.

According to one aspect:
the zone is the RML zone,

$$Eq . 1 is : {π}_{0 , zone} = {c}_{0} ( {( FPF )}^{c 1} \times {( Vent )}^{c 2} \times {( Re )}^{c 3} \times {( \frac{{A}_{branch}}{{A}_{trachea}} )}^{c 4} … \times {π}_{m}^{cm} ) ,$$

and
c1>c2>c3>c4>cm.

According to one aspect:
the zone is the RML zone,

$$Eq . 1 is : {π}_{0 , zone} = {c}_{0} ( {( FPF )}^{c 1} \times {( Vent )}^{c 2} \times {( Re )}^{c 3} \times {( \frac{{A}_{branch}}{{A}_{trachea}} )}^{c 4} ) ,$$

and
c1>c2>c3>c4.

According to one aspect:
the zone is the ET zone,

$$Eq . 1 is : {π}_{0 , zone} = {c}_{0} ( {( FPF )}^{c 1} \times {( \frac{{Q}_{max}}{{Q}_{inh}} )}^{c 2} \times {( LNParam )}^{c 3} \times {( \frac{{L}_{ch}}{MMAD} )}^{c 4} … \times {π}_{m}^{cm} ) ,$$

and
c1>c2>c3>c4>cm.

According to one aspect:
the zone is the ET zone,

$$Eq . 1 is : {π}_{0 , zone} = {c}_{0} ( {( FPF )}^{c 1} \times {( \frac{{Q}_{max}}{{Q}_{inh}} )}^{c 2} \times {( LNParam )}^{c 3} \times {( \frac{{L}_{ch}}{MMAD} )}^{c 4} ) ,$$

and
c1>c2>c3>c4.

According to one aspect:
the zone is the TL zone,

$$Eq . 1 is : {π}_{0 , zone} = {c}_{0} ( {( FPF )}^{c 1} \times {( \frac{{Q}_{max}}{{Q}_{inh}} )}^{c 2} \times {( {V}_{branch , normal} )}^{c 3} \times {( \frac{{L}_{ch}}{MMAD} )}^{c 4} … \times {π}_{m}^{cm} ) ,$$

and
c1>c2>c3>c4>cm.

According to one aspect:
the zone is the TL zone,

$$Eq . 1 is : {π}_{0 , zone} = {c}_{0} ( {( FPF )}^{c 1} \times {( \frac{{Q}_{max}}{{Q}_{inh}} )}^{c 2} \times {( {V}_{branch , normal} )}^{c 3} \times {( \frac{{L}_{ch}}{MMAD} )}^{c 4} ) ,$$

and
c1>c2>c3>c4.

Terms where m>4 are optionally be present. These additional terms are determined the above-mentioned using regression analysis and multiple patient record datasets.

It is understood that the skilled person may include additional steps in order to improve predictive accuracy. For instance, a sensitivity of the dimensionless correlation of a zone to the dimensionless numbers (DN) therein may be determined. This can be achieved by plotting deposition as a function of each DN. Using this technique, “behaviour changing” DN(s) may be found, and also limits (DNₗᵢₘᵢₜ) where the change in the behaviour (change in shape of the plots) occurs. The values in the patient record data set may be divided into two or three groups. The DN_{behaviour} may or may not be one of the DNs in the correlation and may be a combination of at least two DNs. The groups (range) is where DN_{behaviour}<DNₗᵢₘᵢₜ and the other with DN_{behaviour}>DNₗᵢₘᵢₜ, Then, we find correlations for each range separately. Then the accuracy of correlations may be improved and the average maximum ICC for each zone may reach, for instance, 0.95.

Further provided is a method for determining a solved dimensionless correlation, wherein the solved dimensionless correlation is useful for determining airway deposition of an inhaled substance in a lung of a subject (as described herein), the method comprising the steps:
receiving a primary parameter group comprising multiple the parameters from the parameter library of Table B. The primary parameter group may comprise:
   at least 9 parameters from the library of parameters of Table B, and optionally one or more parameters not listed in Table B, or
   the parameters from one of the primary parameter groups of Table A (PPG1 to PPG 5), and optionally one or more parameters not listed in Table B.
performing a dimensionless analysis on the primary parameter group in order to arrive at a set of parameters, and a solved dimensionless correlation, wherein the dimensionless analysis comprises:
   constructing dimensionless numbers from the primary parameter group using Buckingham Pi Theroem, thereby obtaining a dimensionless correlation of the form:

$${π}_{0} = {c}_{0} ( {π}_{1}^{c 1} \times {π}_{2}^{c 2} \times {π}_{3}^{c 3} \times … \times {π}_{k - 1}^{ck - 1} ) \qquad [ Eq 1 ]$$

wherein:
   deposition is represented as dimensionless number π₀,
   c₀ (π₁^{c1}×π₂^{c2}×π₃^{c3}× . . . ×πₖ^{ck-1}) is a dimensionless group,
   each π _{of} π_{1 to k-1} is a dimensionless number,
   each c of c1 to ck−1 is an exponent of the dimensionless number,
   k is a minimum number of dimensionless numbers in the dimensionless correlation, wherein k is (quantity of parameters in the deposition and primary parameter group) minus (number of primary dimensions in the primary parameter group)
   c₀ is a multiplier of the correlation.
solving values for c₀, c1 to cm in a dimensionless correlation:

$${π}_{0} = {c}_{0} ( {π}_{1}^{c 1} \times {π}_{2}^{c 2} \times {π}_{3}^{c 3} \times … \times {π}_{m}^{cm} ) , \qquad [ Eq 1 a ]$$

wherein:
   dimensionless numbers π₁ to πₘ, are a subset of the dimensionless numbers π₁ to πₖ of Eq. 1,
   m has a value at least 4, between 4 and 20, preferably between 4 and 9, more preferably 4, 5 or 6, and is less than k,
   different versions of Eq. 1a containing different of subsets of dimensionless numbers π₁ to πₘ, each subset having a different combination of dimensionless numbers from π₁ to πₖ, are each solved using regression analysis and multiple patient record datasets to obtain values of the multiplier c₀, and exponents c1 to cm,
      wherein:
the solved dimensionless correlation is the version of Eq. 1a containing dimensionless numbers π₁ to πₘ, multiplier c₀, and exponents c1 to cm which best fit the multiple patient records, and
the parameter set comprises the parameters of the solved dimensionless correlation.

The above-mentioned remove and replace protocol is used to reduce the number of dimensionless terms to preferably between 4 and 9, more preferably 4, 5 or 6.

The present method is performed in vitro. The present method is preferably performed in silico. The present method is performed using a computer. The present method is a computer-implemented method.

Further provided is a computing device or system configured for performing the method described herein.

Further provided is a computer program or computer program product having instructions which when executed by a computing device or system cause the computing device or system to perform (each of the steps of) a method as described herein Further provided is a computer readable medium having stored thereon a computer program (product) having instructions which when executed by a computing device or system cause the computing device or system to perform (each of the steps of) a method as described herein.

A data stream which is representative of a computer program or computer program product having instructions which when executed by a computing device or system cause the computing device or system to perform (each of the steps of) the method as described herein.

### Tables

TABLE A
Primary parameter groups (PPG) per zone, parameters within each primary parameter group
(Parameters), dimensionless numbers (DN) determined from the primary parameter group.

| PPG | Zone | Parameters | DN |
|---|---|---|---|
| 1 | NS | IM1 to IM11, IN1 to IN6, | DN1 to DN38 in Table C |
| | | F1 to F10, PC1 to PC12 | |
| | | in Table B | |
| 2 | NS | A_{branch}, A_{trachea}, FPF, GSD, L_{ch}, L_{ch,ref}, MMAD, Qₘₐₓ, Qᵢₙₕ, Rate, tᵢₙⱼ, tᵢₙₕ, TVᵢₙₕ, V_{branch}, V_{branch,total}, V_{TLC}, V_{TLC,total}, V_{FRC}, V_{FRC,total}, u, ρₐ, ρₚ, μₐ, g, Nₒᵤₜₗₑₜ, α, uₚₗᵤₘₑ, vₐ, T_{airway}, Tₐᵢᵣ, k_{d}, d_{nozzle}, tₑₓₕ, s, Tor | $\frac{{A}_{branch}}{{A}_{trachea}} , FPF , \frac{{L}_{ch}}{MMAD} ,$ $\hat{L} , LNParam , \frac{{Q}_{ma x}}{{Q}_{i n h}} ,$ $\frac{Rate \cdot {( {t}_{inj} )}^{2}}{T {V}_{i nh}} , Re , stdLN ,$ \hat{t}, V_{branch,normal}, Vent, $β , St , \frac{{TV}_{inh}}{{V}_{branch}} , R e ,$ |
| | | | $Fr , \frac{{A}_{branch}}{{( {L}_{ch} )}^{2}} , {N}_{outlet} ,$ |
| | | | ${V}_{TLC , n o r mal} , \frac{{V}_{FRC}}{{V}_{TLC}} ,$ |
| | | | $sin ( α ) , \hat{u} , \frac{{V}_{branch}}{{V}_{TLC}} ,$ |
| | | | $Gr , \frac{{A}_{inlet}}{{( {L}_{ch} )}^{2}} , \frac{{L}_{ch}}{MMAD} ,$ |
| | | | $We , Eo , \frac{{d}_{nozzle}^{2}}{{A}_{inlet}} ,$ |
| | | | IE, RR, Br, s, Tor |
| 3 | NS | A_{branch}, A_{trachea}, FPF, GSD, L_{ch}, L_{ch,ref}, MMAD, Qₘₐₓ, Qᵢₙₕ, Rate, tᵢₙⱼ, tᵢₙₕ, TVᵢₙₕ, V_{branch}, V_{branch,total}, V_{TLC}, V_{FRC}, V_{FRC,total}, V_{TLC,total}, u, ρₐ, ρₚ, μₐ, g, Nₒᵤₜₗₑₜ, α, uₚₗᵤₘₑ, T_{airway}, Tₐᵢᵣ, vₐ, σ, k_{d}, d_{nozzle}, tₑₓₕ, | $\frac{{A}_{branch}}{{A}_{trachea}} , FPF ,$ $\frac{{L}_{c h}}{MMAD} , \hat{L} , LNParam ,$ $\frac{{Q}_{m ax}}{{Q}_{i n h}} , \frac{Rate \cdot {t}_{i n j}^{2}}{T {V}_{i n h}} , Re ,$ stdLN, \hat{t}, V_{branch,normal}, $Vent , β , S t , \frac{{TV}_{i n h}}{{V}_{branch}} , Re ,$ |
| | | | $F r , \frac{{A}_{branch}}{{( {L}_{c h} )}^{2}}$ |
| | | | ${N}_{outlet} , {V}_{TLC , normal} , \frac{{V}_{FRC}}{{V}_{TLC}} ,$ |
| | | | $sin ( α ) , \hat{u} , \frac{{V}_{branch}}{{V}_{TLC}} , Gr ,$ |
| | | | $\frac{{A}_{inlet}}{{( {L}_{c h} )}^{2}} , \frac{{L}_{ch}}{MMAD} , We , Eo ,$ |
| | | | $\frac{{( {d}_{nozzle} )}^{2}}{{A}_{inlet}} , IE , RR$ |
| 4 | NS | A_{branch}, A_{trachea}, FPF, GSD, L_{ch}, L_{ch,ref}, MMAD, Qₘₐₓ, Qᵢₙₕ, Rate, tᵢₙⱼ, tᵢₙₕ, TVᵢₙₕ, V_{branch}, V_{branch,total}, V_{TLC}, V_{FRC}, V_{FRC,total}, V_{TLC,total}, u, ρₐ, ρₚ, μₐ g, Nₒᵤₜₗₑₜ, α, uₚₗᵤₘₑ, vₐ | $\frac{{A}_{branch}}{{A}_{trachea}} , F P F ,$ $\frac{{L}_{ch}}{M M A D} , \hat{L} , L N Param ,$ $\frac{{Q}_{max}}{{Q}_{inh}} , \frac{Rate \cdot {( {t}_{inj} )}^{2}}{T {V}_{inh}} ,$ Re, stdLN, \hat{t}, V_{branch,normal}, |
| | | | $Vent , β , St , \frac{T {V}_{inh}}{{V}_{branch}} , Re ,$ |
| | | | $Fr , \frac{{A}_{branch}}{{( {L}_{ch} )}^{2}} , {N}_{outlet} ,$ |
| | | | ${V}_{TLC , normal} , \frac{{V}_{FRC}}{{V}_{TLC}} , sin ( α ) ,$ |
| | | | $\hat{u} , \frac{{V}_{branch}}{{V}_{TLC}}$ |
| 5 | NS | A_{branch}, A_{trachea}, FPF, GSD, L_{ch}, L_{ch,ref}, MMAD, Qₘₐₓ, Qᵢₙₕ, Rate, tᵢₙⱼ, tᵢₙₕ, TVᵢₙₕ, V_{branch}, V_{branch,total}, V_{TLC}, V_{FRC}, V_{FRC,total}, V_{TLC,total}, u, ρₐ, μₐ | $\frac{{A}_{branch}}{{A}_{trachea}} , F P F ,$ $\frac{{L}_{ch}}{M M A D} , \hat{L} , L N Param ,$ $\frac{{Q}_{max}}{{Q}_{inh}} , \frac{Rate \cdot {( {t}_{inj} )}^{2}}{T {V}_{inh}} ,$ |
| | | | Re, stdLN, \hat{t}, |
| | | | V_{branch,normal},Vent, ß |
| 6 | IT | FPF, GSD, L_{ch}, MMAD, Qₘₐₓ, Qᵢₙₕ | $F P F , \frac{{L}_{ch}}{M M A D} ,$ $L N param , \frac{{Q}_{max}}{{Q}_{inh}}$ |
| 7 | ET | FPF, GSD, L_{ch}, MMAD, Qₘₐₓ, Qᵢₙₕ | $F P F , \frac{{L}_{ch}}{M M A D} ,$ $L N param , \frac{{Q}_{max}}{{Q}_{inh}}$ |
| 8 | DI | FPF, GSD, L_{ch}, L_{ch,ref}, MMAD, Qₘₐₓ, Qᵢₙₕ | $F P F , \hat{L} , \frac{{Q}_{max}}{{Q}_{inh}} , stdL N$ |
| 9 | PR | A_{trachea}, FPF, L_{ch}, MMAD, Qₘₐₓ, Qᵢₙₕ, Rate, u, ρₐ, μₐ | $F P F , \frac{{Q}_{max}}{{Q}_{inh}} , Re , β$ |
| 10 | TL | FPF, L_{ch}, MMAD, Qₘₐₓ, Qᵢₙₕ, V_{branch}, V_{branch,total} | $F P F , \frac{{L}_{ch}}{M M A D} ,$ $\frac{{Q}_{max}}{{Q}_{inh}} ,$ V_{branch,normal} |
| 11 | RUL | FPF, Qₘₐₓ, Qᵢₙₕ, V_{TLC}, V_{FRC}, V_{FRC,total}, V_{TLC,total}, V_{branch}, V_{branch,total} | $F P F , \frac{{Q}_{max}}{{Q}_{inh}} , Vent$ V_{branch,normal} |
| 12 | RML | A_{branch}, A_{trachea}, FPF, MMAD, u, V_{TLC}, V_{FRC}, V_{FRC,total}, V_{TLC,total}, ρₐ, μₐ | $\frac{{A}_{branch}}{{A}_{trachea}} , F P F , Re , Vent ,$ |
| 13 | RLL | FPF, L_{ch}, L_{ch,ref}, tᵢₙⱼ, tᵢₙₕ, | FPF, \hat{t}, \hat{t}, |
| | | V_{branch}, V_{branch,total} | V_{branch,normal} |
| 14 | LUL | A_{branch}, A_{trachea}, FPF, L_{ch}, MMAD, Qₘₐₓ, Qᵢₙₕ | $\frac{{A}_{branch}}{{A}_{trachea}} , F P F , \frac{{L}_{ch}}{M M A D} , \frac{{Q}_{max}}{{Q}_{inh}}$ |
| 15 | LLL | FPF, tᵢₙⱼ, Rate, TVᵢₙₕ, V_{branch}, V_{branch,total}, V_{TLC}, V_{FRC}, V_{FRC,total},V_{TLC,total} | $F P F , \frac{Rate \cdot {( {t}_{inj} )}^{2}}{T {V}_{inh}} , Vent ,$ V_{branch,normal} |

Key:
PPG - primary parameter group;
DN - dimensionless number;
NS - not zone specific.
See also keys to Tables B and C.

TABLE A1
PPG 6 to 15 of Table A, wherein the zones are listed (top to bottom) in
order of importance (IT most important), and the DNs of each PPG/zone
are listed in order of predictive power
(1^{st} listed (c₁)> 2^{nd} listed (c₂)> 3^{rd} listed (c₃)> 4^{th} listed (c₄)).

| PPG | Zone | Parameters | DN |
|---|---|---|---|
| 6 | IT | FPF, GSD, L_{ch}, MMAD, Qₘₐₓ, Qᵢₙₕ | $F P F , \frac{{Q}_{max}}{{Q}_{inh}} , L N Param , \frac{{L}_{ch}}{M M A D}$ |
| 9 | PR | A_{trachea}, FPF, L_{ch}, MMAD, Qₘₐₓ, Qᵢₙₕ, Rate, u, ρₐ, μₐ | $F P F , \frac{{Q}_{max}}{{Q}_{inh}} , Re , β$ |
| 8 | DI | FPF, GSD, L_{ch}, L_{ch,ref}, MMAD, Qₘₐₓ, Qᵢₙₕ | $F P F , stdL N , \hat{L} , \frac{{Q}_{max}}{{Q}_{inh}}$ |
| 13 | RLL | FPF, L_{ch}, L_{ch,ref}, | FPF, |
| | | tᵢₙⱼ, tᵢₙₕ, V_{branch}, | V_{branch,normal}, \hat{L}, \hat{t} |
| | | V_{branch,total} | |
| 15 | LLL | FPF, tᵢₙⱼ, Rate, TVᵢₙₕ, V_{branch,zone}, V_{branch,total}, V_{TLC}, V_{FRC}, V_{FRC,total}, V_{TLC,total} | FPF, Vent, V_{branch,normal}, $\frac{Rate \cdot {( {t}_{inj} )}^{2}}{T {V}_{inh}}$ |
| 11 | RUL | FPF, Qₘₐₓ, Qᵢₙₕ, V_{TLC}, V_{FRC}, V_{FRC,total}, V_{TLC,total}, V_{branch}, V_{branch,total} | $F P F , Vent , \frac{{Q}_{max}}{{Q}_{inh}} ,$ V_{branch,normal} |
| 14 | LUL | A_{branch}, A_{trachea}, FPF, L_{ch}, MMAD, Qₘₐₓ, Qᵢₙₕ | $F P F , \frac{{Q}_{max}}{{Q}_{inh}} , \frac{{L}_{ch}}{M M A D} , \frac{{A}_{branch}}{{A}_{trachea}}$ |
| 12 | RML | A_{branch}, A_{trachea}, FPF, MMAD, u, V_{TLC}, V_{FRC}, V_{TLC,total}, ρₐ, μₐ | $F P F , Vent , Re , \frac{{A}_{branch}}{{A}_{trachea}}$ |
| 7 | ET | FPF, GSD, L_{ch}, MMAD, Qₘₐₓ, Qᵢₙₕ | $F P F , \frac{{Q}_{max}}{{Q}_{inh}} , L N Param , \frac{{L}_{ch}}{M M A D}$ |
| 10 | TL | FPF, L_{ch}, MMAD, Qₘₐₓ, Qᵢₙₕ, V_{branch}, V_{branch,total} | $F P F , \frac{{Q}_{max}}{{Q}_{inh}} ,$ V_{branch,normal}, $\frac{{L}_{ch}}{M M A D}$ |

Key:
PPG - primary parameter group;
DN - dimensionless number;
NS - not zone specific.
See also keys to Tables B and C.

TABLE B
Parameter library containing non-exhaustive list of parameters

| Code | Parameter | Abv | ExU | 1° Dim | Subgroup | Comments |
|---|---|---|---|---|---|---|
| IM1 | Zone volume at | V_{TLC} | m³ | L³ | Imaging | Volume of a specified zone at TLC. |
| | TLC | | | | | When the specified zone is a lobe |
| | | | | | | (one of RUL, RML, RLL, LUL, LLL), |
| | | | | | | the volume of that lobe is used. |
| | | | | | | When the specified zone is one of TL, |
| | | | | | | IT, ET, DI, PR, the volume of (RUL + |
| | | | | | | RML + RLL + LUL + LLL) is used. |
| | | | | | | Where V_{TLC, total} is used, it means a |
| | | | | | | sum of all five lobe volumes (RUL + |
| | | | | | | RML + RLL + LUL + LLL) at TLC. |
| IM2 | Zone volume at | V_{FRC} | m³ | L³ | Imaging | Volume of a specified zone at FRC. |
| | FRC | | | | | When the specified zone is a lobe |
| | | | | | | (one of RUL, RML, RLL, LUL, LLL), |
| | | | | | | the volume of that lobe is used. |
| | | | | | | When the specified zone is one of TL, |
| | | | | | | IT, ET, DI, PR, the volume of (RUL + |
| | | | | | | RML + RLL + LUL + LLL) is used. |
| | | | | | | Where V_{FRC, total} is used, it means a |
| | | | | | | sum of volumes of all five lobe (RUL + |
| | | | | | | RML + RLL + LUL + LLL) at FRC. |
| IM3 | Airway surface | A_{branch} | m² | L² | Imaging | Total surface area of airways in a |
| | area at TLC | | | | | specified zone at TLC. |
| | | | | | | When the specified zone is a lobe |
| | | | | | | (one of RUL, RML, RLL, LUL, LLL), |
| | | | | | | the total surface area of airways of |
| | | | | | | that lobe is used. |
| | | | | | | When the specified zone is one of TL, |
| | | | | | | ET, DI, the total surface area of |
| | | | | | | airways of that zone is used. |
| | | | | | | When the specified zone is one of, IT |
| | | | | | | or PR, the combined total surface |
| | | | | | | area of airways of zones CL and DI is |
| | | | | | | used. |
| IM4 | Obstructiveness | A_{min, ua} | m² | L² | Imaging | Minimum transverse cross-sectional |
| | of upper airway | | | | | area of upper airway; the minimum |
| | | | | | | transverse cross-sectional area of |
| | | | | | | upper airway is typically found at the |
| | | | | | | larynx. |
| IM5 | Airway volume | V_{branch} | m³ | L³ | Imaging | Total volume of airways (lumens) in a |
| | at TLC | | | | | specified zone at TLC. |
| | | | | | | When the specified zone is a lobe |
| | | | | | | (one of RUL, RML, RLL, LUL, LLL), |
| | | | | | | the total volume of airways in that |
| | | | | | | lobe is used. |
| | | | | | | When the specified zone is one of TL, |
| | | | | | | ET, DI, the total volume of airways of |
| | | | | | | that zone is used. |
| | | | | | | When the specified zone is one of IT |
| | | | | | | or PR, the combined total volume of |
| | | | | | | airways of zones (CL + DI) is used. |
| | | | | | | Where V_{branch, total} is used, it refers to |
| | | | | | | a combined total of airway (lumens) |
| | | | | | | volumes in the zones (CL + DI). |
| IM6 | Trachea surface | A_{trachea} | m² | L² | Imaging | Transverse cross-sectional area |
| | area at TLC | | | | | perpendicular to the airflow at the |
| | | | | | | start of the trachea, at TLC. |
| IM7 | Number of | Nₒᵤₜₗₑₜ | None | D-less | Imaging | Total number of airway outlets |
| | airways | | | | | present in a specified zone. |
| | | | | | | Where the specified zone is a lobe |
| | | | | | | (one of RUL, RML, RLL, LUL, LLL) |
| | | | | | | the total number of airway outlets |
| | | | | | | present in that lobe is used. |
| | | | | | | Where the specified zone is one of |
| | | | | | | TL, ET, IT, or DI, or PR, the combined |
| | | | | | | total of airway outlets for |
| | | | | | | RUL + RML + RLL + LUL + LLL is used. |
| IM8 | Characteristic | L_{ch} | m | L | Imaging | A length of a specified zone, defined |
| | length | | | | | as IM1 divided by IM3 at that zone. |
| | | | | | | Measured at TLC. |
| | | | | | | Where used, L_{ch, ref} is an average L_{ch} |
| | | | | | | for healthy subjects in the multiple |
| | | | | | | patient record datasets. |
| IM9 | Airway | s | — | D-less | Imaging | A measure of rate of diameter change |
| | smoothness | | | | | in an airway (lumen) of a specified |
| | | | | | | zone in a distal to peripheral direction: |
| | | | | | | average of the fraction of minimum to |
| | | | | | | maximum diameter in an airway |
| | | | | | | (lumen) of the specified zone. |
| | | | | | | Measured at TLC. |
| | | | | | | Where the specified zone is a lobe |
| | | | | | | (one of RUL, RML, RLL, LUL, LLL) |
| | | | | | | the airway smoothness of that lobe is |
| | | | | | | used. |
| | | | | | | Where the specified zone is one of |
| | | | | | | TL, ET, IT, PR, or DI, the average |
| | | | | | | airway smoothness of zone DI is |
| | | | | | | used. |
| IM10 | Airway | Tor | — | D-less | Imaging | Airway tortuosity of a specified zone |
| | tortuosity | | | | | is an average of (ratio of actual |
| | | | | | | airway path length to the straight |
| | | | | | | distance between the ends of the |
| | | | | | | airway) for all airways within the zone, |
| | | | | | | measured at TLC |
| | | | | | | Where the specified zone is a lobe |
| | | | | | | (one of RUL, RML, RLL, LUL, LLL) |
| | | | | | | the airway tortuosity of that lobe is |
| | | | | | | used. |
| | | | | | | Where the specified zone is one of |
| | | | | | | TL, ET, IT, DI, or PR, the average |
| | | | | | | airway tortuosity of all airways in the |
| | | | | | | five lobes is used. same is average of |
| | | | | | | airways in all 5 lobes) |
| IM11 | Airway fractal | FD | — | D-less | Imaging | Is known in the art as self-similarity of |
| | dimension | | | | | an airway over different scales, |
| | | | | | | including branching structures. |
| | | | | | | Measured at TLC. See Moledina S, et |
| | | | | | | al Fractal branching quantifies |
| | | | | | | vascular changes and predicts |
| | | | | | | survival in pulmonary hypertension: a |
| | | | | | | proof of principle study. Heart. 2011 |
| | | | | | | Aug. 1; 97(15): 1245-9. |
| IN1 | Fine particle | FPF | % | D-less | Inhaler | Fraction of particles (by mass) with |
| | fraction | | | | | the aerodynamic size below 5 μm. |
| IN2 | Mass median | MMAD | m | L | Inhaler | Diameter at which 50% (by mass) of |
| | aerodynamic | | | | | the particles of an aerosol with the |
| | diameter | | | | | density of 1000 kg/m³ are larger and |
| | | | | | | 50% are smaller. In terms of meter. |
| IN3 | Geometric | GSD | — | D-less | Inhaler | An indicator of a shape of a log- |
| | Standard | | | | | normal distribution of aerosols. GSD = |
| | Deviation | | | | | exp(σ), where σ is the standard |
| | | | | | | deviation of the natural logarithms of |
| | | | | | | data that have a lognormal |
| | | | | | | distribution, dimensionless. |
| IN4 | Inlet area | Aᵢₙₗₑₜ | m² | L² | Inhaler | Surface area of the inlet of a Dry |
| | | | | | | Powder Inhaler where the |
| | | | | | | surrounding air enters the device and |
| | | | | | | then to the mouth |
| IN5 | Injection | tᵢₙⱼ | s | T | Inhaler | total duration when aerosols are |
| | duration | | | | | exiting an inhaler, in terms of |
| | | | | | | seconds. |
| IN6 | Nozzle | d_{nozzle} | m | L | Inhaler | Diameter of the nozzle of a Metered |
| | diameter in | | | | | dose inhaler |
| | an MDI | | | | | |
| F1 | Mean | Qᵢₙₕ | m³/s | L³/T | Flow | Mean value of flow rate inhaled by a |
| | inhalation | | | | | subject |
| | flow rate | | | | | |
| F2 | Inhaled | TVᵢₙₕ | m³ | L³ | Flow | Total volume of air inhaled by a |
| | volume | | | | | patient during one breath (inhalation) |
| | | | | | | while using the inhaler device |
| F3 | Slope of | Rate | m³/s² | L³/T² | Flow | Slope of an inhalation flow rate profile |
| | rising flow | | | | | at the onset of inhalation. It is an |
| | rate | | | | | indication of how fast a certain |
| | | | | | | volume of air is inhaled. |
| F4 | Peak flow | Qₘₐₓ | m³/s | L³/T | Flow | maximum value of the air flow inhaled |
| | rate | | | | | by a patient during one breath |
| | | | | | | (inhalation) while using the inhaler |
| | | | | | | device |
| F5 | Inhalation | tᵢₙₕ | s | T | Flow | total duration of inhalation of air by a |
| | time | | | | | subject in terms of seconds |
| F6 | Flow velocity | u | m/s | L/T | Flow and | Mean velocity of air at the trachea, |
| | | | | | geometry | which is calculated by dividing F1 by |
| | | | | | | IM6 |
| F7 | Plume angle | α | ° (degree) | D-less | Flow | The angle of the cone covered by a |
| | (for MDIs) | | Sine of | | | plume exiting a Metered Dose Inhaler |
| | | | the angle | | | |
| F8 | Plume velocity | uₚₗᵤₘₑ | m/s | L/T | Flow | Velocity of the plume exiting a |
| | (for MDIs) | | | | | Metered Dose Inhaler |
| F9 | Exhalation | tₑₓₕ | s | T | Flow | total duration of exhalation of air by a |
| | time | | | | | subject in terms of seconds |
| F10 | Respiration | RR | — | D-less | D-less | Quantity of breaths taken per minute |
| | Rate | | | | | |
| PC1 | Density of air | ρₐ | kg/m3 | M/L³ | Physical | Density of air where applicable based |
| | | | | | properties | on the physical meaning of the |
| | | | | | and | dimensionless numbers |
| | | | | | constants | |
| PC2 | Density of | ρₚ | kg/m3 | M/L³ | Physical | Density of particles where applicable |
| | particles | | | | properties | based on the physical meaning of the |
| | | | | | and | dimensionless numbers |
| | | | | | constants | |
| PC3 | Dynamic | μₐ | kg/(ms) | M/(LT) | Physical | The dynamic viscosity of a fluid is the |
| | viscosity of | | | | properties | measure of its resistance to flow |
| | air | | | | and | when an external force is applied. |
| | | | | | constants | |
| PC4 | Kinematic | νₐ | m2/s | L²/T | Physical | The kinematic viscosity is defined as |
| | viscosity of | | | | properties | the absolute viscosity of a liquid |
| | air | | | | and | divided by its density at the same |
| | | | | | constants | temperature. |
| PC5 | Surface | σ | N/m | M/ T² | Physical | Surface tension is the tendency of |
| | tension of | | (kg/s2) | | properties | liquid surfaces at rest to shrink into |
| | droplets (for | | | | and | the minimum surface area possible |
| | MDI) | | | | constants | |
| PC6 | Specific heat | Cp | J/(kg K) | ML²/(T²Θ) | Physical | the quantity of heat absorbed per unit |
| | capacity of air | | | | properties | mass of the material when its |
| | or particles | | | | and | temperature increases 1 K |
| | | | | | constants | |
| PC7 | Enthalpy of | h_{fg} | J/kg | ML²/T² | Physical | amount of energy needed for a liquid |
| | vaporization | | | | properties | substance to transform a gas |
| | | | | | and | |
| | | | | | constants | |
| PC8 | saturated | Pᵥ | Pa | M/(LT²) | Physical | The pressure which the vapor of a |
| | vapour | | | | properties | substance is in equilibrium with the |
| | pressure | | | | and | substance's pure liquid |
| | | | | | constants | |
| PC9 | Thermal | k_{d} | W/(mK) | ML/(T³Θ) | Physical | a measure of the ability of a |
| | conductivity | | | | properties | substance to conduct heat |
| | | | | | and | |
| | | | | | constants | |
| PC10 | gravitational | g | m/s2 | L/ T² | Physical | the acceleration of an object in free |
| | acceleration | | | | properties | fall within a vacuum (and thus without |
| | | | | | and | experiencing drag). |
| | | | | | constants | |
| PC11 | Temperature | T_{airway} | K | Θ | Physical | a physical quantity that expresses the |
| | of airway | | | | properties | perceptions of hotness and coldness. |
| | | | | | and | |
| | | | | | constants | |
| PC12 | Temperature of | Tₐᵢᵣ | K | Θ | Physical | a physical quantity that expresses the |
| | surrounding air | | | | properties | perceptions of hotness and coldness. |
| | | | | | and | |
| | | | | | constants | |

Key: Abv—abbreviation; ExU—exemplary units; 1° Dim—Primary dimension (L, M, T or Θ); TLC—total lung capacity - the lung volume attained after a deep inhalation; FRC—functional residual capacity - lung volume attained after normal expiration. D-less—dimensionless.

TABLE C
Dimensionless numbers (DNs)

| DN | Dimensionless | |
|---|---|---|
| code | number | Category |
| DN1 | Nₒᵤₜₗₑₜ | Imaging |
| DN2 | $\frac{{A}_{branch}}{{( {L}_{ch} )}^{2}}$ | Imaging |
| DN3 | $\frac{{A}_{min , ua}}{{A}_{trachea}}$ | Imaging |
| DN4 | $\frac{{A}_{branch}}{{A}_{trachea}}$ | Imaging |
| DN5 | $\frac{{V}_{branch}}{{V}_{TLC}}$ | Imaging |
| DN6 | $\frac{{V}_{FRC}}{{V}_{TLC}}$ | Imaging |
| DN7 | $\hat{L} = \frac{{L}_{ch}}{\overline{{L}_{ch , ref}}}$ | Imaging |
| DN8 | $Vent = \frac{( {V}_{TLC} - {V}_{FRC} )}{{( {V}_{TLC , total} - {V}_{FRC , total} )}_{all lobes}} \times 100$ | Imaging |
| DN9 | ${V}_{TLC , normal} = \frac{{V}_{TLC}}{{V}_{TLC , total}}$ | Imaging |
| DN10 | ${V}_{branch , normal} = \frac{{V}_{branch}}{{V}_{branch , total}}$ | Imaging |
| DN11 | s | Imaging |
| DN12 | Tor | Imaging |
| DN13 | FD | Imaging |
| DN14 | FPF | Inhaler |
| DN15 | GSD | Inhaler |
| DN16 | sin (α) | Inhaler |
| DN17 | $\frac{Rate \cdot {( {t}_{inj} )}^{2}}{T {V}_{inh}}$ | Flow |
| DN18 | $\frac{{Q}_{max}}{{Q}_{inh}}$ | Flow |
| DN19 | $\frac{{L}_{ch}}{M M A D}$ | Shared |
| DN20 | $\frac{{A}_{inlet}}{{( {L}_{ch} )}^{2}}$ | Shared |
| DN21 | $\frac{T {V}_{inh}}{{V}_{branch}}$ | Shared |
| DN22 | $\hat{t} = \frac{{t}_{inj}}{{t}_{inh}}$ | Shared |
| DN23 | $β = \frac{{ρ}_{a} {A}_{trachea} Rate}{{Q}_{inh} {μ}_{a}}$ | Shared |
| DN24 | $Re = \frac{{ρ}_{a} u {L}_{ch}}{{μ}_{a}}$ | Shared |
| DN25 | $Fr = \frac{u}{\sqrt{g {L}_{ch}}}$ | Shared |
| DN26 | $Ga = \frac{g {L}_{c h}^{3}}{{v}_{a}^{2}}$ | Shared |
| DN27 | $S t = \frac{{ρ}_{p} {MMAD}^{2} {μ}_{a} u}{18 {L}_{c h}}$ | Shared |
| DN28 | $We = \frac{{ρ}_{p} {u}^{2} MMAD}{σ}$ | Shared |
| DN29 | $O h = \frac{We}{Re}$ | Shared |
| DN30 | $Eo = \frac{( {ρ}_{p} - {ρ}_{a} ) {g ( {L}_{ch} )}^{2}}{σ}$ | Shared |
| DN31 | $\hat{u} = \frac{{u}_{p l u m e}}{u}$ | Shared |
| DN32 | $Gr = \frac{g ( {T}_{a i r w a y} - {T}_{a i r} ) {( {L}_{c h} )}^{3}}{{v}_{a}^{2} {T}_{a i r}}$ | Shared |
| DN33 | $B r = \frac{{μ}_{a} {u}^{2}}{{k}_{a} ( {T}_{a i r w a y} - {T}_{a i r} )}$ | Shared |
| DN34 | $\frac{{( {d}_{n o z z l e} )}^{2}}{{A}_{inlet}}$ | Inhaler |
| DN35 | $IE = \frac{{t}_{inh}}{{t}_{exh}}$ | Flow |
| DN36 | RR (Respiration Rate) | Flow |
| DN37 | $stdLN = std ( \frac{MMAD}{G S D} , MMAD , MMAD \times GSD )$ | Inhaler |
| DN38 | LNParam = cdf | Inhaler |
| | (lognorm(MMAD, GSD, d_{cutoff} ) | |

Key:
DN: dimensionless number;
cfd: cumulative distribution function, lognorm: normal logarithmic distribution, d_{cutoff} = cutoff diameter for calculating FPF (5 μm), std: standard deviation.
TL - total lobes, IT - intrathoracic, ET - extrathoracic, CL - central, DI - distal, PR - peripheral, RUL - right upper lobe, RML - right middle lobe, RLL - right lower lobe, LUL - left upper lobe, LLL - left lower lobe.

### Example 1 (RML—PPG12)

Multiple patient record datasets were obtained. Each patient record dataset contained a measured value for each parameter of Table A PPG3 (34 parameters in Table A) for that patient and a value for deposition (π₀) in the RML for that patient. Subsequently, values for the 32 dimensionless numbers of Table A PPG3 were calculated. A selection (random or otherwise) was made of 9 dimensionless numbers from the 32 dimensionless numbers to form a “replaceable set”, and were used in Eq 1a, wherein each of π₁ to π₉ is a dimensionless number.

$${π}_{0 , RML} = {c}_{0} \times {π}_{1}^{c 1} \times {π}_{2}^{c 2} \times {π}_{3}^{c 3} \times … \times {π}_{9}^{c 9} \qquad [ Eq . 1 a ]$$

With the values for each dimensionless number known from the multiple patient record datasets, the multiplier c₀ and the value of each exponent c1 to c9 were solved using the nonlinear least-squares protocol (NLLSP) incorporating Levenberg-Marquardt method. In a first round, initial values of unity (1) were selected for multiplier c₀ and each exponent c1 to c9. The NLLSP adjusted values of (c₀, c1 to c9) iteratively until there was convergence to a solution, providing a first set of values for c₀, c1 to c9. In a second round, three dimensionless numbers with the least impact (lower exponent) were removed from the replaceable set of the first round and a total of three different dimensionless numbers were randomly selected from the 23 remaining dimensionless numbers and added. In the second round, the NLLSP adjusted values for c₀, c1 to c9 until there was convergence to a solution, providing a second set of weight values for c₀, c1 to c9 as determined from the interclass correlation (ICC).

If the ICC was improved in the second round compared with the first round, a third round continued with the replaceable set of the second round, and again 3 dimensionless numbers were removed and three different dimensionless numbers were randomly selected from the 20 remaining dimensionless numbers and added. If the ICC was worse in the second round compared with the first round, a third round continued with the first round. Future rounds proceed as for previous rounds, mutatis mutandis. The process was repeated until insignificant change in ICC. This happened after 500 rounds. The ICC for PPG 12 was 0.86.

Five dimensionless terms of π₁^{c1} to π₉^{c9} were below a significance threshold value and those corresponding dimensionless terms were removed from Eq. 1a, which was resolved into Eq. 1b:

$${π}_{0 , RML} = {c}_{0} \times {( \frac{{A}_{branch}}{{A}_{trachea}} )}^{c 1} \times {FPF}^{c 2} \times {Re}^{c 3} \times {Vent}^{c 4} , \qquad [ Eq . 1 b ]$$

having the same dimensionless numbers as PPG12 (RML) as in Table A.

If the contribution of a dimensionless term was below 0.1% of the total value (p0, RML), then it was removed. The reduced set of dimensionless terms contained four dimensionless terms.

The values of c₀, c1 to c4 for Eq. 1b are set out in Table D.

TABLE D
values of c₀, c1 to c4 for Eq. 1b, determined
from multiple patient record datasets.

| Exponent/constant | Value |
|---|---|
| c₀ | 0.00166 |
| c1 | 0.104 |
| c2 | 1.185 |
| c3 | −0.120 |
| c4 | 0.839 |

Eq. 1b is an example of an equation for determining of RML deposition in a general population.

A patient whose RML deposition using the present method was unknown, had parameters of Table A1 PPG12 (RML) measured/determined, and from these parameters values, values for the dimensionless numbers of Table A PPG12 were determined. The results are shown in Table E, where Col. B are the measured DN (π) values for the patient, and Col. C is the value of the DN in Eq 1b with the weighting of Table D applied.

TABLE E
values of c₀, c1 to c4 for Eq. 1b, determined from multiple patient
record datasets

| A | B | C |
|---|---|---|
| πᵢ in Eq. 1 | Patient value πᵢ | Patient value πᵢ |
| $\frac{{A}_{b r a n c h}}{{A}_{trachea}}$ | $\frac{{A}_{b r a n c h}}{{A}_{trachea}} = 7.622$ | 1.235 |
| FPF | FPF = 62.2 | 133.54 |
| Re | Re = 0.0027 | 2.033 |
| Vent | Vent = 6.162 | 4.598 |

The patent values were applied to Eq. 1b, viz:

$${π}_{0 , RML} = 0.00166 \times 1.235 \times 133.54 \times 2.033 \times 4.598 = 2.56 = deposition in RML .$$

The deposition in RML as predicted using CFD as 2.7. The accuracy of the present method was determined as 94.53%.

### Example 2

19 patients whose RML deposition using the present method was unknown, each had parameters of Table A1 PPG12 (RML) measured/determined, and therefrom values of RML deposition determined according to Eq. 1b of Example 1. The results (depPredicted [%]) are shown in Table F. Prediction using the present method compares well with deposition determined using CFD (depCFD [%])—mean values 2.56 vs 2.26 respectively (90.3% agreement).

TABLE F
RML deposition in RML of each of 19 subjects as determine by CFD
(depCFD[%]) and the present method (depPredicted[%]).

| | depCFD[%] | depPredicted[%] |
|---|---|---|
| Patient1 | 2.19 | 2.56 |
| Patient2 | 3.64 | 3.03 |
| Patient3 | 2.71 | 2.51 |
| Patient4 | 2.66 | 2.37 |
| Patient5 | 2.61 | 2.72 |
| Patient6 | 2.88 | 2.23 |
| Patient7 | 1.26 | 2.50 |
| Patient8 | 1.71 | 2.43 |
| Patient9 | 1.49 | 2.62 |
| Patient10 | 1.32 | 2.46 |
| Patient11 | 1.70 | 3.01 |
| Patient12 | 3.71 | 2.87 |
| Patient13 | 0.21 | 1.77 |
| Patient14 | 2.18 | 2.68 |
| Patient15 | 4.66 | 2.51 |
| Patient16 | 1.98 | 2.48 |
| Patient17 | 2.04 | 2.61 |
| Patient18 | 2.47 | 2.31 |
| Patient19 | 1.58 | 2.91 |
| Mean | 2.26 | 2.56 |
| STD | 0.99 | 0.29 |

### Example 3

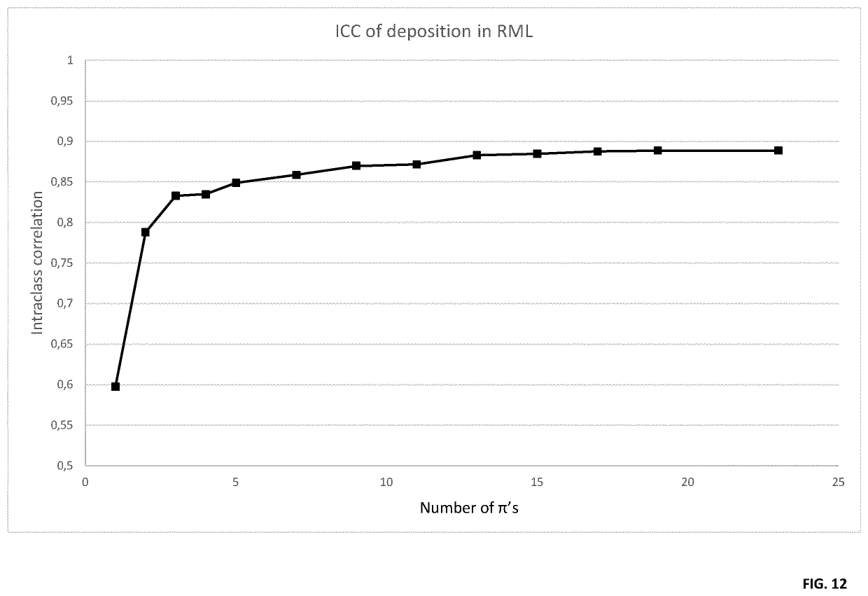

Regression analysis was compared with different quantities of π's in Eq. 1a in Example 1. Values of interclass correlation were determined when Eq. 1a contain only one π (π₁^{c1}) and when the quantities of π's increased incrementally until Eq. 1a contained π₁^{c1} to π₂₃^{c23}. The results are shown in FIG. 12; the interclass correlation does not significantly improve beyond four π terms in Eq. 1b, meaning that a minimum of 4 dimensionless numbers can be used to predict deposition in each zone.

### Example 4

A primary parameter group was formed containing all the parameters of PPG3 of Table A. Using dimensional analysis and Buckingham Pi Theorem, dimensionless numbers were constructed.

For each parameter in the primary parameter group, its primary dimension(s) was determined i.e. M (mass), L (length), t (time) and/or Θ (temperature). The number (j) of primary dimensions in the primary parameter group was determined (4 primary dimensions M,L,T, Θ). The number (n) of parameters in the deposition (De) and primary parameter group was determined. Based on PPG3 of Table A=34 parameters. The minimum number (k) of dimensionless numbers (k=n−j) in the deposition and primary parameter group was determined as 34−4=30.

The dimensionless numbers (π) were written out as:

$${π}_{0} = Deposition , and$$

$${π}_{1} , {π}_{2} , {π}_{3} , … , {π}_{30}$$

Four repeating variables (parameters) were selected, of which three related to mass (ρₐ), geometry (L_{ch} (Characteristic length, IM8)) and kinematics (u (Flow velocity, F6)), and the fourth was the injection time (tᵢₙⱼ, IN5) (see Table B for parameter codes). To construct a dimensionless number (π₁), the holding expression comprised the repeating variables and one remaining parameter which was dynamic viscosity of air (μₐ(PC2) in Table B).

Thus:

$${π}_{i} = {ρ}_{a}^{A 1} \times {L}_{ch}^{B 1} \times {u}^{C 1} \times {t}^{D 1} \times {μ}_{a}^{1}$$

Their dimensions were:

$${L}^{0} {M}^{0} {T}^{0} = {( {M}^{1} {L}^{- 3} )}^{A 1} \times {( {L}^{1} )}^{B 1} \times {( {L}^{1} {T}^{- 1} )}^{C 1} \times {Θ}^{D 1} \times ( {M}^{1} {L}^{- 1} {T}^{- 1} )$$

$${L}^{0} {M}^{0} {T}^{0} = {( L )}^{- 3 A 1 + B 1 + C 1 - 1} \times {( T )}^{- C 1 - 1} \times {( M )}^{A 1 + 1} \times {( Θ )}^{D 1}$$

$$\{ \begin{matrix}L : 0 = - 3 A 1 + B 1 + C 1 - 1 \\ T : - C 1 - 1 \to C 1 = - 1 \\ M : 0 = A 1 + 1 \to A 1 = - 1 \\ θ : 0 = D 1\end{matrix} \to 0 = 3 + B 1 - 1 - 1 \to B 1 = - 1$$

$${π}_{1} = {ρ}_{a}^{- 1} \times {L}_{ch}^{- 1} \times {u}^{- 1} \times {t}^{0} \times {μ}_{a}^{1}$$

Re-arranging the above,

$${π}_{1} = \frac{{μ}_{a}}{{ρ}_{a} {uL}_{ch}} ,$$

which is equivalent to the Reynolds number (inverse form) and was denoted as DN23 (Table C).

To construct the next dimensionless number (π₂), the holding expression comprised the same repeating variables, and one “remaining parameter” was chosen which was g (PC10). After application of the Buckingham Pi theorem, as exemplified for π₁ above, the π₂ was determined as:

$${π}_{2} = Fr = \frac{u}{\sqrt{{gL}_{ch}}}$$

denoted as DN25 (Table C)

Some dimensionless numbers were created by dividing two parameters having the same primary dimensions. Other were created by repeating the above Buckingham Pi theorem process. In total 32 dimensionless numbers were found are shown in Table A, PPG3.

The dimensionless numbers were placed in the dimensionless correlation

$${π}_{0} = {c}_{0} \times {π}_{1}^{c 1} \times {π}_{2}^{c 2} \times {π}_{3}^{c 3} \times … \times {π}_{m}^{cm} \qquad [ Eq . 1 a ]$$

in which a selection of m dimensionless numbers was made from the 32 dimensionless numbers found using the Buckingham Pi Theorem.

Initially, a random selection was made of 9 dimensionless numbers from the 32 dimensionless numbers (replaceable set), and multiplier c₀ and the value of each exponent c1 to c9 were solved using a nonlinear least-squares protocol (NLLSP) incorporating Levenberg-Marquardt method and the multiple patient record datasets In a first round, initial values of unity (1) were selected for multiplier c₀ and each exponent c1 to c9. The NLLSP adjusted values of (c₀, c1 to c9) iteratively until there was convergence to a solution, providing a first set of values for c₀, c1 to c9. Convergence to a solution meant that, when applying the multiple patient record datasets to the dimensionless correlation (Eq. 1a), the value of π₀ obtained approached the record value of deposition. Typically, convergence to a solution was reached within a certain number of iterations. The interclass correlation (ICC) was determined (typically above 0.8).

In a second round, a total of three dimensionless numbers were randomly removed from the selection (replaceable set) of the first round and a total of three different dimensionless numbers were randomly selected from the remaining (32−9=23) dimensionless numbers and added. In the second round, the NLLSP adjusted values for c₀, c1 to c9 until there was convergence to a solution, providing a second set of weight values for c₀, c1 to c9.

The interclass correlation (ICC) was determined (typically above 0.8) If the ICC was improved in the second round compared with the first round, a third round continued with the dimensionless numbers (replaceable set) of the second round, and again 3 dimensionless numbers were removed and three different dimensionless numbers were randomly selected from the remaining (32−9−3=20) dimensionless numbers and added. If the ICC was worse in the second round compared with the first round, a third round continued with the first round. Future rounds proceed as for previous rounds, mutatis mutandis. The process was repeated until insignificant change in ICC. This typically happened after 500 rounds. The average ICC overall was 0.92.

In this way, the dimensionless correlation was reached having a reduced number of dimensionless numbers. It was found that having more than four dimensionless numbers did not improve predictive power of deposition. One dimensionless correlation was determined for each zone.

The dimensionless numbers determined for each zone are shown in Table A, PPG6 to PPG15. Dimensionless numbers found most frequently as determinants for deposition across all zones and dimensionless numbers found less frequently but have a higher weighting (exponent) are shown in Table A, PPG2 and PPG3. Dimensionless numbers found most frequently as determinants for deposition across almost all zones are shown in Table A, PPG4. Dimensionless numbers found most frequently as determinants for deposition across most zones are shown in Table A, PPG5. Dimensionless numbers found as determinants for deposition in zones IT, ET, DI, PR, TL, RUL, RML, RLL, RUL, LUL, LLL is shown in Table A, PPG5 to PPG15 respectively. Table A1 shows the four most determining dimensionless numbers for deposition in zones IT, ET, DI, PR, TL, RUL, RML, RLL, RUL, LUL, LLL in PPG5 to PPG15.

After determining a dimensionless correlation for each zone, multiple unused patient record (test) datasets were applied to each dimensionless correlation. Each unused patient record dataset was not used in the regression analysis.

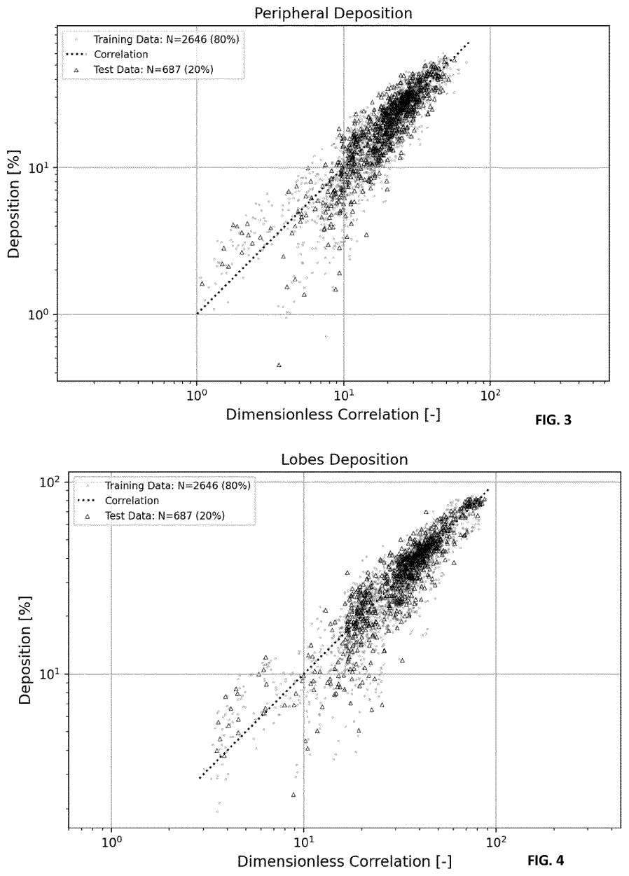

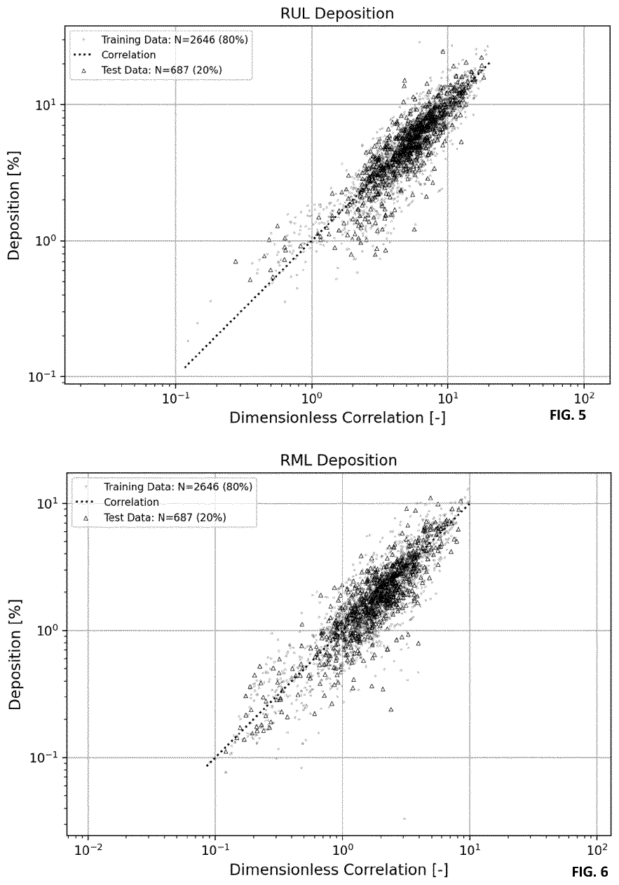

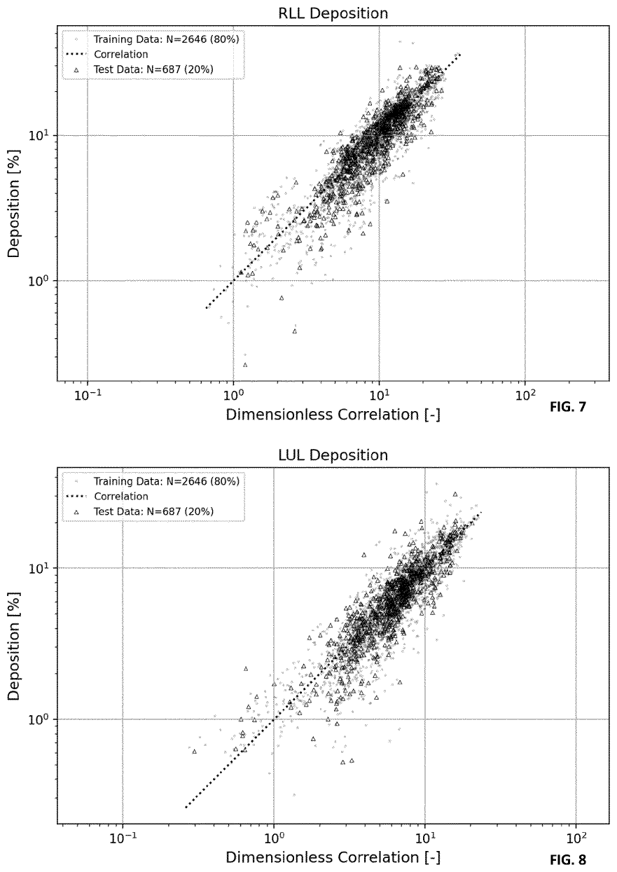

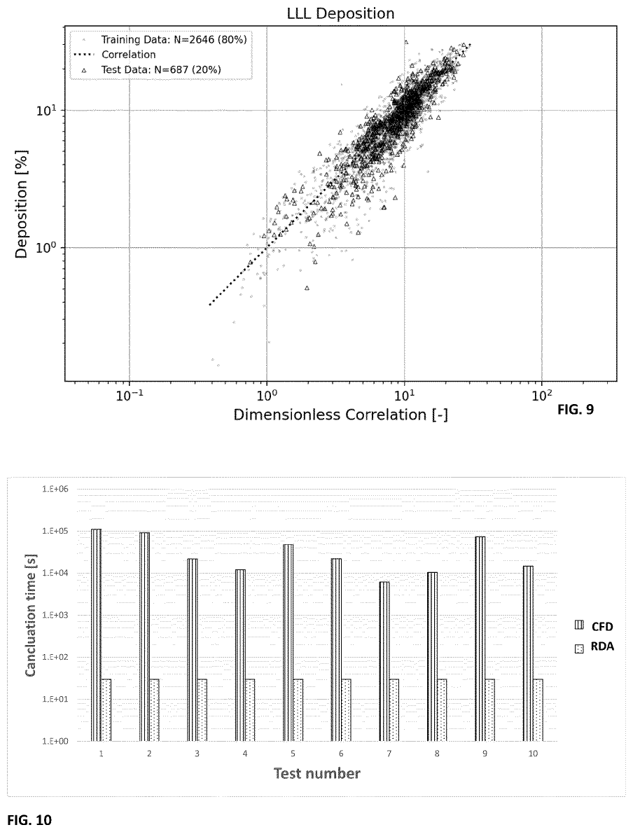

A dimensionless correlation per zone demonstrated a good fit to the training data (IT zone), FIG. 2 (DI zone), FIG. 3 (PR zone), FIG. 4 (TL zone), FIG. 5 (RUL zone), FIG. 6 (RML zone), FIG. 7 (RLL zone), FIG. 8 (LUL zone), FIG. 9 (LLL zone)). Note that results for the extrathoracic zone are not shown because they are linked to intrathoracic data (extrathoracic is a remainder after extrathoracic has been substracted).

The results demonstrate good fit of the dimensionless correlation to the training data per zone, and also a good fit of the test data to the dimensionless correlation per zone. Table G shows interclass correlations (ICC) for the training data and test data of each of FIGS. 1 to 9. An ICC of greater than 0.7 is considered a good fit.

TABLE G
Interclass correlations (ICC) for the training
data and test data of each of FIGs. 1 to 9.

| | IT | DI | PR | TL | RUL | RML | RLL | LUL | LLL |
|---|---|---|---|---|---|---|---|---|---|
| FIG. | 1 | 2 | 3 | 4 | 5 | 6 | 7 | 8 | 9 |
| ICC | 0.92 | 0.91 | 0.88 | 0.92 | 0.87 | 0.86 | 0.88 | 0.86 | 0.89 |
| training | | | | | | | | | |
| ICC test | 0.92 | 0.91 | 0.88 | 0.92 | 0.86 | 0.83 | 0.88 | 0.86 | 0.89 |

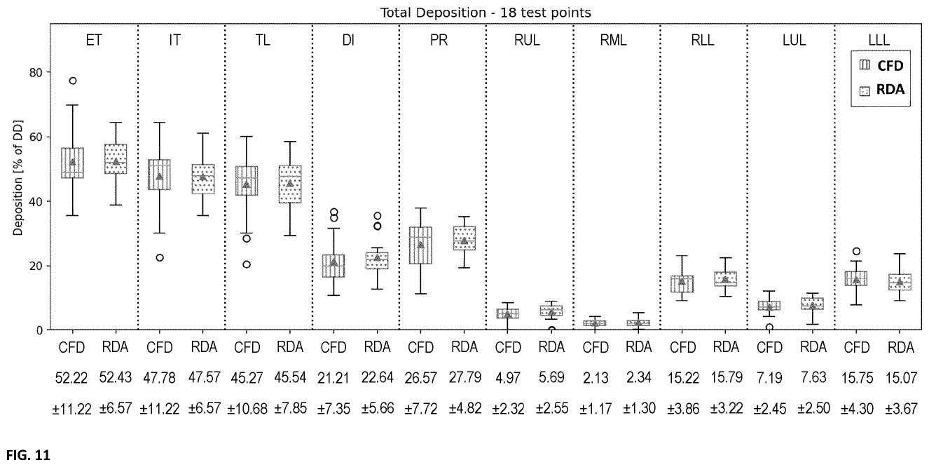

For a sample of 18 patients using the parameter set (each separately imaged, same inhaler/drug (IN parameters), same inhalation profile (F numbers), same physical properties and constants (PC parameters), deposition obtained by the presently-described dimensionless correlation (NDA) is similar to the deposition obtained by Computation fluid dynamics (CFD) (see average values and standard deviations in FIG. 11).

Calculation time of deposition using the presently-described dimensionless correlation (NDA) is many times faster than using Computation fluid dynamics (CFD) to achieve a similar levels of accuracy (see FIG. 10). Computation time using NDA was around 30 seconds on average, compared with 42 000 seconds (11.5 hours) on average for CFD.

### Example 5

Depositions in each of the extrathoracic (ET), intrathoracic (IT), distal (DI), peripheral (PR), right upper lobe (RUL), right middle lobe (RML), right lower lobe (RLL), left upper lobe (LUL), left lower lobe (LLL) zones were determined using the present method (RDA) for 19 different patients. These were compared with depositions determined from the same 19 patients using CFD.

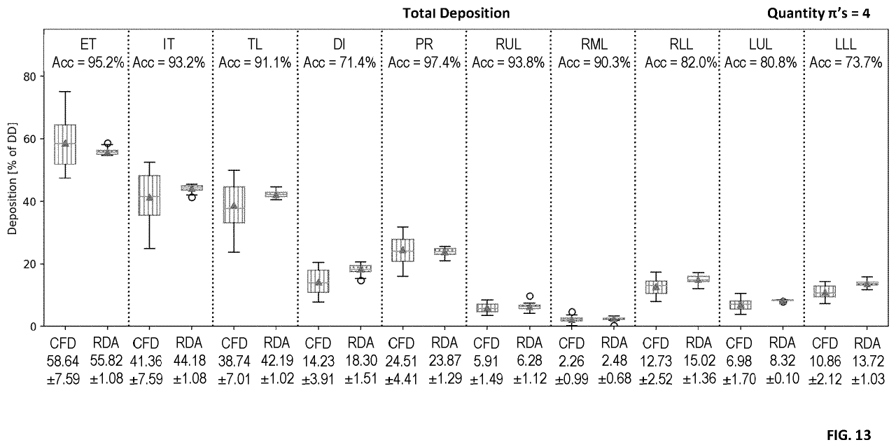

FIG. 13 shows the averaged results when the quantity of π's in Eq. 1 was 4. For each zone, the spread of depositions as calculated using CFD and RDA is indicated as a bar with error lines, the mean value, the standard deviation, and the accuracy of RDA compared with CFD.

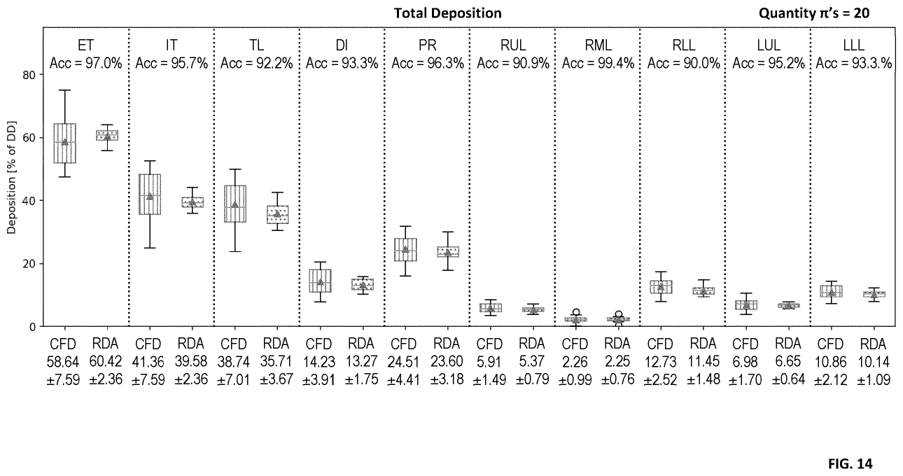

FIG. 14 is similar to FIG. 13, except the quantity of π's in Eq. 1 was 20.

The results demonstrate high accuracy of the present method compared with CFD, and a insignificant increase in accuracy when the number of π's in Eq. 1 is raised from 4 to 20.

1. A computer implemented method for determining airway deposition of an inhaled substance in a zone of a lung of a subject, comprising:
receiving a patient dataset of the subject comprising a parameter set of two or more parameters and corresponding values, wherein the parameters in the parameter set form at least four dimensionless numbers in a dimensionless correlation specific to the zone,
determining, from the dimensionless correlation and the corresponding values of the parameters in the parameter set, the airway deposition of the inhaled substance in the zone of the lung of the subject.

2. The method according to claim 1, wherein:
a first component of the dimensionless correlation is a dimensionless number (π_{0,zone}) representative of the airway deposition for the zone being determined, and
a second component of the dimensionless correlation is a dimensionless group comprising the at least four dimensionless numbers for the zone being determined.

3. The method according to claim 1, wherein the dimensionless correlation has a form of Eq. 1:
$${π}_{0 , zone} = {c}_{0} ( {π}_{1}^{c 1} \times {π}_{2}^{c 2} \times {π}_{3}^{c 3} \times … \times {π}_{m}^{cm} ) \qquad [ Eq . 1 ]$$
where
π_{0,zone} is a dimensionless number that is deposition in the zone (first component),
c₀ (π₁^{c2}×π₂^{c2}×π₃^{c3}× . . . ×πₘ^{cm}) is the dimensionless group (second component),
each π _{of} π_{1 to m} is a dimensionless number for the zone,
each c of c1 to cm is an exponent of the dimensionless number,
m is at least 4, between 4 and 20, preferably between 4 and 9, more preferably 4, 5 or 6,
c₀ is a multiplier of the correlation,
each of m of π_{1 to} πₘ, each c of c1 to cm, and c₀ for the zone is determined from multiple patient record datasets and regression analysis.

4. The method according to claim 3, wherein each of π of π₁ to πₘ, each c of c1 to cm, and c₀ for the zone is determined by:
populating the dimensionless group of Eq. 1 with 6 or more, preferably 6 to 20 dimensionless numbers selected from a pool of dimensionless numbers,
performing multiple rounds regression analysis, wherein each round comprises:
adjusting values for c₀, c1 to cm until there is convergence to a solution for deposition for the multiple patient record datasets,
substituting a portion (e.g. 2 to 5) of the dimensionless numbers in the dimensionless group with different dimensionless numbers selected from the pool of dimensionless numbers, and
repeating the adjusting and substituting until improvement in convergence (interclass correlation) is minimal.

5. The method according to claim 4, wherein the pool of dimensionless numbers comprises:
$$\frac{{A}_{branch}}{{A}_{trachea}} , F P F , \frac{{L}_{ch}}{M M A D} , \hat{L} , L N Param , \frac{{Q}_{max}}{{Q}_{inh}} , \frac{Rate \cdot {( {t}_{inj} )}^{2}}{T {V}_{inh}} , Re , stdL N , \hat{t} , {V}_{branch , normal} , Vent , β , St , \frac{T {V}_{inh}}{{V}_{branch}} , Re , Fr , \frac{{A}_{branch}}{{( {L}_{ch} )}^{2}} , {N}_{outlet} , {V}_{TLC , normal} , \frac{{V}_{FRC}}{{V}_{TLC}} , sin ( α ) , \hat{u} , \frac{{V}_{branch}}{{V}_{TLC}} , Gr , \frac{{A}_{inlet}}{{( {L}_{ch} )}^{2}} , \frac{{L}_{ch}}{M M A D} , We , Eo , \frac{{d}_{nozzle}^{2}}{{A}_{inlet}} , I E , R R , Br , s , and Tor .$$

6. The method according to claim 3, wherein the dimensionless numbers of Eq. 1 are selected from:
$$\frac{{A}_{branch}}{{A}_{trachea}} , F P F , \frac{{L}_{ch}}{M M A D} , \hat{L} , L N Param , \frac{{Q}_{max}}{{Q}_{inh}} , \frac{Rate \cdot {( {t}_{inj} )}^{2}}{T {V}_{inh}} , Re , stdL N , \hat{t} , {V}_{branch , normal} , Vent , and β .$$

7. The method according claim 1, wherein the parameters in the parameter set and the dimensionless numbers comprise those of Table AA1, selected according to the zone of the lung of the subject where airway deposition is to be determined:
Table AA1

| Zone | Parameters | DN |
|---|---|---|
| Intrathoracic, IT | FPF, GSD, L_{ch}, MMAD, Qₘₐₓ, Qᵢₙₕ | $FPF , \frac{{Q}_{max}}{{Q}_{inh}} , LNParam , \frac{{L}_{ch}}{MMAD}$ |
| Peripheral, PR | A_{trachea}, FPF, L_{ch}, MMAD, Qₘₐₓ, Qᵢₙₕ, Rate, u, ρₐ, μₐ | $FPF , \frac{{Q}_{max}}{{Q}_{inh}} , Re , β$ |
| Distal, DI | FPF, GSD, L_{ch}, L_{ch,ref}, MMAD, Qₘₐₓ, Qᵢₙₕ | $FPF , stdLN , \hat{L} , \frac{{Q}_{max}}{{Q}_{inh}}$ |
| Right lower lobe, RLL | FPF, L_{ch}, L_{ch,ref}, tᵢₙⱼ, tᵢₙₕ, V_{branch}, V_{branch,total} | FPF, V_{branch,normal}, \hat{L}, \hat{t} |
| Left lower lobe, LLL | FPF, tᵢₙⱼ, Rate, TVᵢₙₕ, V_{branch,zone}, V_{branch,total}, V_{TLC}, V_{FRC}, V_{FRC,total}, V_{TLC,total} | $FPF , Vent , {V}_{branch , normal} , \frac{Rate \cdot {( {t}_{inj} )}^{2}}{{TV}_{inh}}$ |
| Right upper lobe, RUL | FPF, Qₘₐₓ, Qᵢₙₕ, V_{TLC}, V_{FRC}, V_{FRC,total}, V_{TLC,total}, V_{branch}, V_{branch,total} | $FPF , Vent , \frac{{Q}_{max}}{{Q}_{inh}} , {V}_{branch , normal}$ |
| Left upper lobe, LUL | A_{branch}, A_{trachea}, FPF, L_{ch}, MMAD, Qₘₐₓ, Qᵢₙₕ | $FPF , \frac{{Q}_{max}}{{Q}_{inh}} , \frac{{L}_{ch}}{MMAD} , \frac{{A}_{branch}}{{A}_{trachea}}$ |
| Right middle lobe, RML | A_{branch}, A_{trachea}, FPF, MMAD, u, V_{TLC}, V_{FRC}, V_{TLC,total}, ρₐ, μₐ | $FPF , Vent , Re , \frac{{A}_{branch}}{{A}_{trachea}}$ |
| Extrathoracic, ET | FPF, GSD, L_{ch}, MMAD, Qₘₐₓ, Qᵢₙₕ | $FPF , \frac{{Q}_{max}}{{Q}_{inh}} , LNParam , \frac{{L}_{ch}}{MMAD}$ |
| Total lobes, TL | FPF, L_{ch}, MMAD, Qₘₐₓ, Qᵢₙₕ, V_{branch}, V_{branch,total} | $FPF , \frac{{Q}_{max}}{{Q}_{inh}} , {V}_{branch , normal} , \frac{{L}_{ch}}{MMAD}$ |

8. The method according to claim 3, wherein:
the zone is the IT zone,
$$Eq . 1 is : {π}_{0 , zone} = {c}_{0} ( {( F P F )}^{c 1} \times {( \frac{{Q}_{max}}{{Q}_{inh}} )}^{c 2} \times {( L N Param )}^{c 3} \times {( \frac{{L}_{ch}}{M M A D} )}^{c 4} ... \times {π}_{m}^{cm} ) ,$$
c1>c2>c3>c4>cm, wherein terms where m>4 are optionally present.

9. The method according to claim 3, wherein:
the zone is the PR zone,
$$Eq . 1 is : {π}_{0 , zone} = {c}_{0} ( {( F P F )}^{c 1} \times {( \frac{{Q}_{max}}{{Q}_{inh}} )}^{c 2} \times {( Re )}^{c 3} \times {( β )}^{c 4} ... \times {π}_{m}^{cm} ) ,$$
c1>c2>c3>c4>cm, wherein terms where m>4 are optionally present.

10. The method according to claim 3, wherein:
the zone is the DI zone,
$$Eq . 1 is : {π}_{0 , zone} = {c}_{0} ( {( F P F )}^{c 1} \times {( stdL N )}^{c 2} \times {( \hat{L} )}^{c 3} \times {( \frac{{Q}_{max}}{{Q}_{inh}} )}^{c 4} ... \times {π}_{m}^{cm} ) ,$$
and
c1>c2>c3>c4>cm, wherein terms where m>4 are optionally present.

11. The method according to claim 3, wherein
the zone is the RUL zone,
$$Eq . 1 is : {π}_{0 , zone} = {c}_{0} ( {( F P F , )}^{c 1} \times {( {V}_{branch , normal} )}^{c 2} \times {( \hat{L} )}^{c 3} \times {( \hat{t} )}^{c 4} ... \times {π}_{m}^{cm} ) ,$$
and
c1>c2>c3>c4>cm, wherein terms where m>4 are optionally present.

12. The method according to claim 3, wherein
the zone is the LUL zone,
$$Eq . 1 is : {π}_{0 , zone} = {c}_{0} ( {( F P F )}^{c 1} \times {( Vent )}^{c 2} \times {( {V}_{branch , normal} )}^{c 3} \times {( \frac{Rate \cdot {( {t}_{inj} )}^{2}}{T {V}_{inh}} )}^{c 4} ... \times {π}_{m}^{cm} ) ,$$
and
c1>c2>c3>c4>cm, wherein terms where m>4 are optionally present.
or
the zone is the RML zone,
$$Eq . 1 is : {π}_{0 , zone} = {c}_{0} ( {( F P F )}^{c 1} \times {( Vent )}^{c 2} \times {( \frac{{Q}_{max}}{{Q}_{inh}} )}^{c 3} \times {( {V}_{branch , normal} )}^{c 4} ... \times {π}_{m}^{cm} ) ,$$
and
c1>c2>c3>c4>cm, wherein terms where m>4 are optionally present.
or
the zone is the RML zone,
$$Eq . 1 is : {π}_{0 , zone} = {c}_{0} ( {( F P F )}^{c 1} \times {( \frac{{Q}_{max}}{{Q}_{inh}} )}^{c 2} \times {( \frac{{L}_{ch}}{M M A D} )}^{c 3} \times {( \frac{{A}_{branch}}{{A}_{trachea}} )}^{c 4} ... \times {π}_{m}^{cm} ) ,$$
and
c1>c2>c3>c4>cm, wherein terms where m>4 are optionally present.
or
the zone is the RML zone,
$$Eq . 1 is : {π}_{0 , zone} = {c}_{0} ( {( F P F )}^{c 1} \times {( Vent )}^{c 2} \times {( Re )}^{c 3} \times {( \frac{{A}_{branch}}{{A}_{trachea}} )}^{c 4} ... \times {π}_{m}^{cm} ) ,$$
and
c1>c2>c3>c4>cm, wherein terms where m>4 are optionally present.
or
the zone is the ET zone,
$$Eq . 1 is : {π}_{0 , zone} = {c}_{0} ( {( F P F )}^{c 1} \times {( \frac{{Q}_{max}}{{Q}_{inh}} )}^{c 2} \times {( L N Param )}^{c 3} \times {( \frac{{L}_{ch}}{M M A D} )}^{c 4} ... \times {π}_{m}^{cm} ) ,$$
and
c1>c2>c3>c4>cm, wherein terms where m>4 are optionally present.
or
the zone is the TL zone,
$$Eq . 1 is : {π}_{0 , zone} = {c}_{0} ( {( F P F )}^{c 1} \times {( \frac{{Q}_{max}}{{Q}_{inh}} )}^{c 2} \times {( {V}_{branch , normal} )}^{c 3} \times {( \frac{{L}_{ch}}{M M A D} )}^{c 4} ... \times {π}_{m}^{cm} ) ,$$
and
c1>c2>c3>c4>cm, wherein terms where m>4 are optionally present.

13. A computing device or system configured for performing the method according to claim 1.

14. A computer program or computer program product having instructions which when executed by a computing device or system cause the computing device or system to perform a method according to claim 1.

15. The method according to claim 2, wherein the dimensionless correlation has a form of Eq. 1:
$${π}_{0 , zone} = {c}_{0} ( {π}_{1}^{c 1} \times {π}_{2}^{c 2} \times {π}_{3}^{c 3} \times ... \times {π}_{m}^{cm} ) \qquad [ Eq . 1 ]$$
where
π_{0,zone} is a dimensionless number that is deposition in the zone (first component),
c₀ (π₁^{c1}×π₂^{c2}×π₃^{c2}× . . . ×πₘ^{cm}) is the dimensionless group (second component),
each π _{of} π_{1 to m} is a dimensionless number for the zone,
each c of c1 to cm is an exponent of the dimensionless number,
m is at least 4, between 4 and 20, preferably between 4 and 9, more preferably 4, 5 or 6,
c₀ is a multiplier of the correlation,
each of π of π₁ to πₘ, each c of c1 to cm, and c₀ for the zone is determined from multiple patient record datasets and regression analysis.

16. The method according to claim 4, wherein the dimensionless numbers of Eq. 1 are selected from:
$$\frac{{A}_{branch}}{{A}_{trachea}} , F P F , \frac{{L}_{ch}}{M M A D} , \hat{L} , L N Param , \frac{{Q}_{max}}{{Q}_{inh}} , \frac{Rate \cdot {( {t}_{inj} )}^{2}}{T {V}_{inh}} , Re , stdL N , \hat{t} , {V}_{branch , normal} , Vent , and β .$$

17. The method according to claim 5, wherein the dimensionless numbers of Eq. 1 are selected from:
$$\frac{{A}_{branch}}{{A}_{trachea}} , F P F , \frac{{L}_{ch}}{M M A D} , \hat{L} , L N Param , \frac{{Q}_{max}}{{Q}_{inh}} , \frac{Rate \cdot {( {t}_{inj} )}^{2}}{T {V}_{inh}} , Re , stdL N , \hat{t} , {V}_{branch , normal} , Vent , and β .$$

18. The method according to claim 2, wherein the parameters in the parameter set and the dimensionless numbers comprise those of Table AA1, selected according to the zone of the lung of the subject where airway deposition is to be determined:
Table AA1

| Zone | Parameters | DN |
|---|---|---|
| Intrathoracic, IT | FPF, GSD, L_{ch}, MMAD, Qₘₐₓ, Qᵢₙₕ | $FPF , \frac{{Q}_{max}}{{Q}_{inh}} , LNParam , \frac{{L}_{ch}}{MMAD}$ |
| Peripheral, PR | A_{trachea}, FPF, L_{ch}, MMAD, Qₘₐₓ, Qᵢₙₕ, Rate, u, ρₐ, μₐ | $FPF , \frac{{Q}_{max}}{{Q}_{inh}} , Re , β$ |
| Distal, DI | FPF, GSD, L_{ch}, L_{ch,ref}, MMAD, Qₘₐₓ, Qᵢₙₕ | $FPF , stdLN , \hat{L} , \frac{{Q}_{max}}{{Q}_{inh}}$ |
| Right lower | FPF, L_{ch,} L_{ch,ref}, | FPF, V_{branch,normal}, \hat{L}, \hat{t} |
| lobe, RLL | tᵢₙⱼ, tᵢₙₕ, V_{branch}, | |
| | V_{branch,total} | |
| Left lower lobe, LLL | FPF, tᵢₙⱼ, Rate, TVᵢₙₕ, V_{branch,zone}, V_{branch,total}, V_{TLC},V_{FRC}, V_{FRC,total}, V_{TLC,total} | $FPF , Vent , {V}_{branch , normal} , \frac{Rate \cdot {( {t}_{inj} )}^{2}}{{TV}_{inh}}$ |
| Right upper lobe, RUL | FPF, Qₘₐₓ, Qᵢₙₕ, V_{TLC}, V_{FRC}, V_{FRC,total}, V_{TLC,total}, V_{branch}, V_{branch,total} | $FPF , Vent , \frac{{Q}_{max}}{{Q}_{inh}} , {V}_{branch , normal}$ |
| Left upper lobe, LUL | A_{branch}, A_{trachea}, FPF, L_{ch}, MMAD, Qₘₐₓ, Qᵢₙₕ | $FPF , \frac{{Q}_{max}}{{Q}_{inh}} , \frac{{L}_{ch}}{MMAD} , \frac{{A}_{branch}}{{A}_{trachea}}$ |
| Right middle lobe, RML | A_{branch}, A_{trachea}, FPF, MMAD, u, V_{TLC}, V_{FRC}, V_{TLC,total}, ρₐ, μₐ | $FPF , Vent , Re , \frac{{A}_{branch}}{{A}_{trachea}}$ |
| Extra- thoracic, ET | FPF, GSD, L_{ch}, MMAD, Qₘₐₓ, Qᵢₙₕ | $FPF , \frac{{Q}_{max}}{{Q}_{inh}} , LNParam , \frac{{L}_{ch}}{MMAD}$ |
| Total lobes, TL | FPF, L_{ch}, MMAD, Qₘₐₓ, Qᵢₙₕ, V_{branch}, V_{branch,total} | $FPF , \frac{{Q}_{max}}{{Q}_{inh}} , {V}_{branch , normal} , \frac{{L}_{ch}}{MMAD}$ |

19. The method according to claim 3, wherein the parameters in the parameter set and the dimensionless numbers comprise those of Table AA1, selected according to the zone of the lung of the subject where airway deposition is to be determined:
Table AA1

| Zone | Parameters | DN |
|---|---|---|
| Intrathoracic, IT | FPF, GSD, L_{ch}, MMAD, Qₘₐₓ, Qᵢₙₕ | $FPF , \frac{{Q}_{max}}{{Q}_{inh}} , LNParam , \frac{{L}_{ch}}{MMAD}$ |
| Peripheral, PR | A_{trachea}, FPF, L_{ch}, MMAD, Qₘₐₓ, Qᵢₙₕ, Rate, u, ρₐ, μₐ | $FPF , \frac{{Q}_{max}}{{Q}_{inh}} , Re , β$ |
| Distal, DI | FPF, GSD, L_{ch}, L_{ch,ref} , MMAD, Qₘₐₓ, Qᵢₙₕ | $FPF , stdLN , \hat{L} , \frac{{Q}_{max}}{{Q}_{inh}}$ |
| Right lower lobe, RLL | FPF, L_{ch}, L_{ch,ref}, tᵢₙⱼ, tᵢₙₕ, V_{branch}, V_{branch,total} | FPF, V_{branch,normal}, \hat{L}, \hat{t} |
| Left lower lobe, LLL | FPF, tᵢₙⱼ, Rate, TVᵢₙₕ, V_{branch,zone}, V_{branch,total}, V_{TLC}, V_{FRC}, V_{FRC,total}, V_{TLC,total} | $FPF , Vent , {V}_{branch , normal} , \frac{Rate \cdot {( {t}_{inj} )}^{2}}{{TV}_{inh}}$ |
| Right upper lobe, RUL | FPF, Qₘₐₓ, Qᵢₙₕ, V_{TLC}, V_{FRC}, V_{FRC,total}, V_{TLC,total}, V_{branch}, V_{branch,total} | $FPF , Vent , \frac{{Q}_{max}}{{Q}_{inh}} , {V}_{branch , normal}$ |
| Left upper lobe, LUL | A_{branch}, A_{trachea}, FPF, L_{ch}, MMAD, Qₘₐₓ, Qᵢₙₕ | $FPF , \frac{{Q}_{max}}{{Q}_{inh}} , \frac{{L}_{ch}}{MMAD} , \frac{{A}_{branch}}{{A}_{trachea}}$ |
| Right middle lobe, RML | A_{branch}, A_{trachea}, FPF, MMAD, u, V_{TLC}, V_{FRC}, V_{TLC,total}, ρₐ, μₐ | $FPF , Vent , Re , \frac{{A}_{branch}}{{A}_{trachea}}$ |
| Extrathoracic, ET | FPF, GSD, L_{ch}, MMAD, Qₘₐₓ, Qᵢₙₕ | $FPF , \frac{{Q}_{max}}{{Q}_{inh}} , LNParam , \frac{{L}_{ch}}{MMAD}$ |
| Total lobes, TL | FPF, L_{ch}, MMAD, Qₘₐₓ, Qᵢₙₕ, V_{branch}, V_{branch,total} | $FPF , \frac{{Q}_{max}}{{Q}_{inh}} , {V}_{branch , normal} , \frac{{L}_{ch}}{MMAD}$ |

20. The method according to claim 4, wherein the parameters in the parameter set and the dimensionless numbers comprise those of Table AA1, selected according to the zone of the lung of the subject where airway deposition is to be determined:
Table AA1

| Zone | Parameters | DN |
|---|---|---|
| Intrathoracic, IT | FPF, GSD, L_{ch}, MMAD, Qₘₐₓ, Qᵢₙₕ | $FPF , \frac{{Q}_{max}}{{Q}_{inh}} , LNParam , \frac{{L}_{ch}}{MMAD}$ |
| Peripheral, PR | A_{trachea}, FPF, L_{ch}, MMAD, Qₘₐₓ, Qᵢₙₕ, Rate, u, ρₐ, μₐ | $FPF , \frac{{Q}_{max}}{{Q}_{inh}} , Re , β$ |
| Distal, DI | FPF, GSD, L_{ch}, L_{ch,ref}, MMAD, Qₘₐₓ, Qᵢₙₕ | $FPF , stdLN , \hat{L} , \frac{{Q}_{max}}{{Q}_{inh}}$ |
| Right lower lobe, RLL | FPF, L_{ch}, L_{ch,ref}, tᵢₙⱼ, tᵢₙₕ, V_{branch}, V_{branch,total} | $FPF , {V}_{branch , normal} , \hat{L} , \hat{t}$ |
| Left lower lobe, LLL | FPF, tᵢₙⱼ, Rate, TVᵢₙₕ, V_{branch,zone}, V_{branch,total}, V_{TLC}, V_{FRC}, V_{FRC,total}, V_{TLC,total} | $FPF , Vent , {V}_{branch , normal} , \frac{Rate \cdot {( {t}_{inj} )}^{2}}{{TV}_{inh}}$ |
| Right upper lobe, RUL | FPF, Qₘₐₓ, Qᵢₙₕ, V_{TLC}, V_{FRC}, V_{FRC,total}, V_{TLC,total}, V_{branch}, V_{branch,total} | $FPF , Vent , \frac{{Q}_{max}}{{Q}_{inh}} , {V}_{branch , normal}$ |
| Left upper lobe, LUL | A_{branch}, A_{trachea}, FPF, L_{ch}, MMAD, Qₘₐₓ, Qᵢₙₕ | $FPF , \frac{{Q}_{max}}{{Q}_{inh}} , \frac{{L}_{ch}}{MMAD} , \frac{{A}_{branch}}{{A}_{trachea}}$ |
| Right middle lobe, RML | A_{branch}, A_{trachea}, FPF, MMAD, u, V_{TLC}, V_{FRC}, V_{TLC,total}, ρₐ, μₐ | $FPF , Vent , Re , \frac{{A}_{branch}}{{A}_{trachea}}$ |
| Extrathoracic, ET | FPF, GSD, L_{ch}, MMAD, Qₘₐₓ, Qᵢₙₕ | $FPF , \frac{{Q}_{max}}{{Q}_{inh}} , LNParam , \frac{{L}_{ch}}{MMAD}$ |
| Total lobes, TL | FPF, L_{ch}, MMAD, Qₘₐₓ, Qᵢₙₕ, V_{branch}, V_{branch,total} | $FPF , \frac{{Q}_{max}}{{Q}_{inh}} , {V}_{branch , normal} , \frac{{L}_{ch}}{MMAD}$ |
